(19) United States
(12) Patent Application Publication
(10) Pub. No.: US 2014/0120106 A1
(43) Pub. Date: May 1, 2014

(54) Stimulation of Arterial Collateral Growth and Lymphogenesis

(71) Applicant: YALE UNIVERSITY, New Haven, CT (US)

(72) Inventors: Michael Simons, Hamden, CT (US); Yong Deng, Branford, CT (US)

(21) Appl. No.: 14/131,163

(22) PCT Filed: Jul. 6, 2012
(86) PCT No.: PCT/US2012/045853

§ 371 Date: Jan. 6, 2014

Related U.S. Application Data
(60) Provisional application No. 61/504,889, filed on Jul. 6, 2011.

(51) Int. Cl. C07C 323/32; C07K 16/28; C07K 14/705

(52) U.S. Cl. CPC C07C 323/32; C07K 14/705; C07K 16/28
USPC 424/139.1; 558/396; 514/523; 536/24.5; 530/387.9; 514/44 A; 536/23.1

(57) ABSTRACT

Compositions and method for stimulating and controlling arteriogenesis and lymphatic vasculature by preventing and/or reducing the cellular interaction between RAF1 and AKT have been developed. The compositions include molecules that increase the bioavailability of non-phosphorylated RAF1, for example, the RAF1 Ser259 to Ala259 mutant in (RAF1 S259A), and AKT1 inhibitory molecules. Defects, disorders or diseases of insufficient blood or lymphatic vasculature are treated by administering to a patient in need thereof, a pharmaceutical composition comprising a molecule specifically blocking RAF1-AKT crosstalk in a pharmaceutically acceptable carrier or excipient in an amount effective to enhance the growth of blood or lymphatic vasculature in the patient. Compositions can be administered by injection or by controlled or sustained release devices, coating on devices or implants, microparticles, bulking agents or depots, or other techniques providing controlled or sustain release over a period of time effective to induce blood or lymphatic vasculature growth as desired.

### CROSS-REFERENCE TO RELATED APPLICATIONS

This application is a 371 application of International Application No. PCT/US2012/045853 entitled “Stimulation of Arterial Collateral Growth and Lymphogenesis, filed in the United States Receiving Office for the Patent Cooperation Treaty on Jul. 6, 2012, which claims the benefit of and priority to U.S. Provisional Application No. 61/504,889, filed on Jul. 6, 2011, the disclosures of which are incorporated herein by reference.

### STATEMENT REGARDING FEDERALLY SPONSORED RESEARCH OR DEVELOPMENT

This invention was made with government support under Agreement Nos. R01 HL053793, R01 HL084619 and R01 HL062289 awarded to Michael Simons by the National Institutes of Health. The government has certain rights in the invention.

### REFERENCE TO SEQUENCE LISTING

The Sequence Listing submitted Jan. 6, 2014 as a text file named “YU_{—}5298_ST25.txt,” created on Dec. 27, 2013, and having a size of 5,686 bytes is hereby incorporated by reference pursuant to 37 C.F.R. §1.52(e)(5).

### FIELD OF THE INVENTION

The present invention relates generally to the modulation of neovascularization and/or the growth of collateral arteries or other arteries from preexisting arteriolar connections.

### BACKGROUND OF THE INVENTION

Arteriogenesis is a process of arterial vasculature formation that occurs both during embryonic development and in adult tissues (Simons, Methods Enzymol, 445:331-342 (2008)). During embryonic development, arteriogenesis involves coating of newly formed arterial endothelial tubes by pericytes and, eventually, smooth muscle cells (Carmeliet, Nat Med, 6:389-395 (2000); Simons, J. Am. Coll. Cardiol., 46:835-837 (2005)). In adult tissues, arteriogenesis can occur by either remodeling of pre-existing collateral arteries or by de novo arterial formation, most likely by arterialization of a subset of capillary vasculature (Carmeliet, Nat Med, 6:389-395 (2000); Simons, Methods Enzymol, 445:331-342 (2008)).

Angiogenesis refers to the formation of capillary networks. Arteriogenesis refers to the growth of preexistent collateral arterioles leading to formation of large conductance arteries that are well capable to compensate for the loss of function of occluded arteries. These preexistent arterioles are present in both the coronary and the peripheral circulation. In fact the presence of these preexistent collateral connections was first reported from Oxford University in 1669. There, the English anatomist Richard Lower observed the following; “Coronary vessels describe a circular course to ensure a better general distribution, and encircle and surround the base of the heart. From such an origin they are able to go off, respectively to opposite regions of the heart, yet around the extremities they come together again and here and there communicate by anastomoses. As a result fluid injected into one of them spreads at one and the same time through both. There is everywhere an equally great need of vital heat and nourishment, so deficiency of these is very fully guarded against by such anastomoses”. Thus, this English researcher not only observed very precisely the presence of preexistent collateral connections between different vascular regions, but actually already recognized their function as alternative pathways for blood flow in case of flow deficiency.

In contrast to the preexistent nature of these collateral vessels, their presence in pathological conditions of obstructive arterial disease was never disputed. In 1971 it was shown for the first time that preexistent collateral arterioles develop into large collateral arteries via proliferation of endothelial and smooth muscle cells and that collateral vessel growth is not simple vasodilatation. Moreover, the dispute about the functionality of collateral arteries was ended by a series of studies relating the extent of their development to outcome after myocardial infarction. In these studies it was definitely shown that “collateral arteries save tissue and life”.

Coronary artery disease is still the most frequent cause of death in the Western world. Outside the Western world, the number of patients with coronary artery disease or peripheral vascular disease is increasing rapidly. Current options to treat occlusive arterial disease include medical therapy or revascularization techniques such as percutaneous transluminal angioplasty (PTCA or PTA) or bypass surgery. These techniques have been developed over the last decades and can be performed nowadays with low morbidity and mortality in patients with chronic coronary artery disease. However, a large number of patients remain for whom this kind of therapy is not feasible, either primarily or after non-successful PTA-PTCA or bypass-surgery, and for many patients outside the industrialized world it is unaffordable. Moreover, the increased survival of patients with acute coronary syndromes, treated medically or with revascularization techniques, leads to an increase in the number of patients with chronic arterial disease. The stimulation of collateral artery growth (arteriogenesis) and/or capillary network growth (angiogenesis) would be of potential benefit to these patients.

Several strategies have been tested for their potential to stimulate the process of arteriogenesis. These strategies focus either on shear stress, direct stimulation of endothelial and smooth muscle cell growth or at the monocytic pathway. The increased shear stress leads to an upregulation of cell adhesion molecules for circulating monocytes, which accumulate around the proliferating arteries and provide cytokines and growth factors. However, some important questions remain to be answered before arteriogenesis can be brought from bench to bedside.

For example, potential candidates for arteriogenic therapy are patients at a progressive stage of their disease. Therefore, unlike the experimental models, their collateral circulation has been remodeled already for a long time period. Nevertheless, these patients remain symptomatic in spite of maximal growth of the collateral circulation. Whether such mature vessels remain responsive to arteriogenic therapy remains unknown.

While VEGF signaling is thought to play a central role in arterial morphogenesis, the precise sequence of events remains undefined. This is of critical importance as therapeutic attempts relying on treatment with VEGF or other angiogenic growth factors have failed in clinical trials, largely due to resistance of diseased endothelium to growth factor stimulation Simons, Cir., 111(12):1556-1566 (2005); Simons, et al., Cir., 102(11):E73-86 (2000).

There is still a need for effective methods of stimulating arteriogenesis.

The lymphatic system is composed of a vascular network of thin-walled capillaries that drain protein-rich lymph from the extracellular spaces within most organs. A continuous single-cell layer of overlapping endothelial cells lines the lymphatic capillaries, which lack a continuous basement membrane and are, therefore, highly permeable. Lymph returns to venous circulation via the larger lymphatic collecting vessels, which contain a muscular and adventitial layer, and the thoracic duct. The lymphatic system also includes lymphoid organs such as the lymph nodes, tonsils, Peyer's patches, spleen, and thymus, all of which play an important role in the immune response. The network of blind-ended, thin-walled capillaries and larger vessels that drain protein-rich interstitial fluid from the extracellular spaces plays a critical role in fluid regulation, immune response and tumor metastasis. Defects in the lymphatic system, congenital as well as acquired, are encountered in a number of disease states (see Alitalo, Nature 438:946-953 (2005); Schulte-Merker, J Cell Biol 193:607-618 (2011)).

One of the more common and least understood lymphatic defects is lymphangiectasia, a pathological dilation of dysmorphic lymphatic vasculature that can lead to lymphedema and compression of nearby structures (Faul, Am J Respir Crit Care Med 161:1037-1046 (2000); Adams, et al. Nat Rev Mol Cell Biol 8:464-478 (2007)). Lymphatic defects such as lymphangiectasia can be particularly prominent in patients with Noonan and LEOPARD syndromes, conditions characterized by gain-of-function mutations in the RAS/RAF signaling cascade (Aoki, et al. Hum Mutat 29:992-1006 (2007); Tidyman, et al. Curr Opin Genet Dev 19:230-236.5, 6 (2009)). The molecular basis of the lymphatic defects in these diseases is still unknown.

Under normal conditions, PI3K/Akt signaling pathway inhibits Erk signaling via Akt1-dependent phosphorylation of Raf1 on Ser259 in EC (Ren, et al. J Clin Invest 120:1217-1228. (2010)). In humans, a Ser259 to Ala259 mutation of RAF1 has been frequently identified in Noonan syndrome patients.

Mammalian lymphatic vessels originate from embryonic veins (Oliver, Nat Rev Immunol 4:35-45 (2004); Srinivasan, Genes Dev 21:2422-2432 (2007)). During early embryonic development, a subset of PROX1-positive EC forms at E10.5 in the lateral portion of the cardinal veins. These cells then sprout laterally, starting at E11.5, to form lymph sacs. Prox1 knockout embryos lack lymph sacs and lymphatic vessels (Wigle, Cell 98:769-778 (1999)) and Prox1-deficient endothelial cells (EC) fail to express lymphatic endothelial markers and instead retain their blood vascular endothelial phenotype. At later stages of development, PROX1 expression is reduced in veins and becomes restricted to the lymphatic vasculature Francois, et al. Physiology (Bethesda) 26:146-155.(2011).

The homeobox transcription factor SOX18 is transiently expressed in cardinal vein EC prior to PROX1, and is required for initiation of the lymphatic EC (LEC) differentiation program upstream of PROX1. During LEC fate induction, SOX18 expression is not restricted to venous EC but it is also expressed in arterial EC, which do not continue to express PROX1 (Francois, Nature 456:643-647 (2008)). In contrast to PROX1, SOX18 expression in the lymphatic vasculature is not detected during later stages of embryonic lymphangiogenesis, suggesting that SOX18 does not play a role in the maintenance of LEC identity. Two closely related Group F Sox factors, SOX7 and SOX17, are able to functionally substitute for SOX18 in vitro and in vivo in a strain-dependent manner (Hosking, Development 136:2385-239113 (2009)). However, neither of these factors is normally expressed during lymphatic development. Sox18 null embryos show a complete lack of PROX1-positive cells and of LEC differentiation from the cardinal vein. Despite the critical role SOX18 plays in developmental lymphangiogenesis, nothing is known about molecular mechanisms controlling its expression.

It is therefore an object of the present invention to provide compositions for stimulating arterial collateral growth.

It is also an object of the present invention to provide a method for stimulating arterial collateral growth.

It is still an object of the present invention to provide a method for treating or ameliorating the symptoms in a patient in need thereof, by stimulating arterial collateral growth.

It is another object of the present invention to provide compositions and methods for stimulating lymphatic vasculature growth.

### SUMMARY OF THE INVENTION

Compositions and method for stimulating and controlling arteriogenesis and lymphatic vasculature have been developed. The compositions and methods stimulate arteriogenesis by preventing and/or reducing the cellular interaction between RAF1 and AKT. The compositions include molecules that increase the bioavailability of non-phosphorylated RAF1, for example, the RAF1 Ser259 to Ala259 mutant in (RAF1 S259A), and AKT1 inhibitory molecules. The method of stimulating arteriogenesis may include reducing AKT1 bioavailability. In a preferred embodiment, the bioavailability of non-phosphorylated RAF1 is increased by introducing a Ser259 to Ala259 mutation in Raf1 (RAF1 S259A), administering a RAF1 S259A protein or polypeptide or a nucleic acid encoding RAF1 S259A to the subject. In other embodiments, the method includes reducing the bioavailability of AKT1 and/or preventing the interaction of Raf1 with AKT1 using, for example, small molecules or antibodies specific for AKT1 which prevent phosphorylation of the RAF1. A constitutively expressed ERK can also be used, if formulation or delivered to a site where the blood or lymphatic vasculature is needed.

Defects, disorders or diseases of insufficient blood or lymphatic vasculature are treated by administering to a patient in need thereof, a pharmaceutical composition comprising a molecule specifically blocking RAF1-AKTcrosstalk in a pharmaceutically acceptable carrier or excipient in an amount effective to enhance the growth of blood or lymphatic vasculature in the patient. The methods are used to stimulate arteriogenesis or growth of lymphatic vasculature in subjects in need thereof, for example, in patients with defects, disorders or diseases of insufficient blood and lymphatic vasculature, for example, advanced vascular diseases such as atherosclerosis, diabetes or other conditions associated with defective arterial development or arterial insufficiency such as advanced coronary, peripheral or cerebral artery diseases and ischemic cardiomyopathy. Compositions can be administered by injection or by controlled or sustained release devices, coating on devices or implants, microparticles, bulking agents or depots, or other techniques providing controlled or sustain release over a period of time effective to induce blood or lymphatic vasculature growth as desired.

### BRIEF DESCRIPTION OF THE DRAWINGS

FIGS. 1A-1E are Western blots showing blocking of RAFT-AKT crosstalk caused ERK constitutive activation in endothelial cells. FIG. 1A, BAEC cells were treated with 10 μM LY294002 or DMSO for 30 minutes. Activation of ERK and AKT was analyzed by western blot with indicated antibodies. FIG. 1B, BAEC cells serum-starved for overnight were pretreated either with DMSO or 10 μM LY294002 for 30 minutes. The cells were then stimulated with 50 ng/ml VEGF for indicated times and activation of ERK and AKT was analyzed by western blot with indicated antibodies. FIG. 1C, HUAEC cells were transfected with 5 nmol control scramble, Akt1 and Akt2 siRNAs. Forty eight hours later, the cells were serum starved for overnight and stimulated with 50 ng/ml VEGF for indicated times and analyzed by western blot with indicated antibodies. FIG. 1D, HUVEC cells were infected with equal amount of empty control (Control), RAF1 WT (Raf1 WT) or RAF1 S259A (Raf1 S259A) lentivirus. Forty eight hours later, the cells were selected with 0.5 μg/ml puromycin for 4 days and analyzed by western blot with indicated antibodies. FIG. 1E, HUVEC cells described above were lysed as described in Materials and Methods, and immunoprecipitation was performed using anti-HA or control mouse IgG. The immunoprecipitates were then analyzed by western blot with indicated antibodies.

FIG. 2A is a bar graph showing quantification of tube length from a Matrigel tube formation assay, in cells infected with WT RAF1, RAF1 S259A compared to control (empty lentivirus). FIG. 2B is a graph showing cell proliferation of cells infected with WT RAF1, RAF1 S259A compared to control (empty lentivirus). FIG. 2C is a graph showing cell migration in cells infected with WT RAF1, RAF1 S259A compared to control (empty lentivirus), in PBS, FBS and VEGF analyzed with wound healing assay. FIG. 2D, apoptosis assay. Standard errors were all calculated based on three independent experiments.

FIGS. 3A to 3M show expression of Dll4 (FIG. 3A), Hey 1 (FIG. 3B), Hey 2 (FIG. 3C), Hes 1 (FIG. 3D), Coup-TFII (FIG. 3E), EphB4 (FIG. 3F), Dll1 (FIG. 3G), Ephrin B2 (FIG. 3G), Notch 1 (FIG. 3I), Notch 4 (FIG. 3J), Jagged 1 (FIG. 3K), FLK1 (FIG. 3L), Nrp1 (3N) and Flt4 (3N). FIG. 3O is a western blot of lysates from HUVECs infected with empty lentivirus (control cells), Raf1 WT lentivirus, and Raf1 S259A. FIG. 3P is a western blot of cell lysates from HUVEC cells were infected with different doses (MOI: 0, 5, 10, 50) of Raf1 WT or S259A adenovirus for 48 hours. FIG. 3Q shows Dll4 expression in AKT^{+/+} and AKT^{-/-} cells expressing WT RAF1, RAF1 S259A compared to control.

FIGS. 4A to 4D show the effect of Raf1SA introduction on Sox18 (FIG. 4A), Sox 7 (FIG. 4B), Ets1 FIG. 4C) and Egr1 (FIG. 4D). FIGS. 4E-4G show the effect of introducing of the Ad-RAF1S259A construct into the femoral artery, on Dll4, Ephrin B2 and Hey 2 expression.

FIGS. 5A and 5B are graphs showing Dll4 (FIG. 5A) and Eprin B2 (FIG. 5B) gene expression in HUVECs infected with control, Raf1 WT, Raf1 S259A lentivirus and treated with 10 μM/ml U0126, 10 μM LY294002 or equal volume of DMSO. FIG. 5C is a Western Blot showing Dll4 protein levels in HUVEC cell infected with control, Raf1 WT, Raf1 S259A lentivirus and treated with 10 μM/ml U0126, 10 μM LY294002 or equal volume of DMSO. FIG. 5D is a bar graph showing Dll4 gene expression in HUVEC cells were treated with 2 and 10 μM U0126, 2 and 10 μM LY294002 or equal volume of DMSO. FIG. 5E is a bar graph showing Dll4 expression in HUVECs infected with lacZ, ME or LA adenovirus at MOI 50 and 100. FIG. 5F is a Western Blot from cell lysates of HUVECs infected with lacZ, ME or LA adenovirus at MOI 50 and 100.

FIGS. 6A-6D are figures showing the generation of a mouse model of Noonan syndrome by endothelial-expression of RAF1S259A. FIG. 6A is a scheme of construct for TRE-RAF1S259A transgenic mice. FIGS. 6B and 6C are graphs of expression of transgenic human RAF1S259A (FIG. 6A) and endogenous mouse Raf1 (FIG. 6B) in purified primary EC from E12.5 wild type (WT), VE-cadherin-tTA (tTA), TRE-RAF1S259A (TRE-S259A) and VE-cadherin-tTA/TRE-RAF1S259A (tTA/TRE-S259A) embryos analyzed by qPCR. hRAF1S259A was specifically expressed in tTA/TRE-S259A embryos, while endogenous mRaf1 was expressed at the same level in all of the embryos.

FIG. 7A is a graph of S259A mouse developing lymphangiectasia, providing a graph of the quantitative analysis of lymphatic vessel diameter (white dashed lines) based on VEGFR3 staining (Control, n=5; S259A, n=3) RAF1S259A does not affect LEC proliferation. LEC proliferation rate in E12.5 (FIG. 7B) and E14.5 (FIG. 7C) embryos was calculated as percentage of Ki67 and PROX1 double positive cells versus total PROX1 positive cells

FIGS. 8A-8H are graphs showing RAF1S259A induction of lymphatic endothelial fate specification. qPCR analysis of Prox1 expression in lentivirus-transduced HUVEC (FIG. 8A) and HUAEC (FIG. 8B) and adenovirus-transduced HDLEC (FIG. 8C). Mean±SEM, n=3. Expression of Vegfr3 and Lyve1 in HUVECs (FIGS. 8D and 8E) and HDLECs (FIGS. 8F and 8G) infected with indicated lentiviruses (FIGS. 8D and 8F) and adenoviruses (FIGS. 8E and 8F) respectively was assessed by qPCR. Both Vegfr3 and Lyve1 were up-regulated. Data are Mean±SEM (n=3). Vegfr3 expression in primary ECs isolated from E12.5 embryos analyzed by qPCR (FIG. 8H). Data are Mean±SEM (n=5).

FIGS. 9A-9G are graphs of RAF1S259A induction of SOX18-initiated lymphatic endothelial fate specification. Expression of Sox18 (FIG. 9A), Sox17 (FIG. 9B), and Sox7 (FIG. 9C) in HUVEC transduced with a null (control), wild type Raf1 (WT) or RAF1S259A (S259A) lentiviruses determined by qPCR. Mean±SEM, n=3. qPCR analysis of Sox18 (FIG. 9D) and Sox17 (FIG. 9E) expression of HDLEC infected with adenoviruses expressing GFP, wild type Raf1 (WT) or RAF1S259A (S259A) constructs. Mean±SEM, n=3. qPCR analysis of Sox18 expression in primary EC isolated from E12.5 embryos (FIG. 9F). Mean±SEM, n=5. FIG. 9G shows quantification of lumen area of cardinal vein. Data Mean±SEM (n=4).

FIG. 9H shows that RAF1S259A does not affect COUP-TFII expression. Immunofluorescence staining of COUP-TFII revealed no difference in COUP-TFII expression between S259A and control embryos in either jugular veins or lymph sacs at E12.5. Immunofluorescence staining of COUP-TFII and SOX18 revealed no difference in COUP-TFII expression between S259A and control embryos in either jugular veins or lymph sacs at E14.5, while SOX18 level was higher in S259A embryos compared to the control embryos. Coup-TFII expression in HUVECs infected with empty control, Raf1 WT and S259A lentiviruses was analyzed by qPCR. Data are means±s.e. of three replicates.

FIGS. 10A-10H show that RAF1-AKT crosstalk regulates lymphatic endothelial fate specification. FIG. 10A shows serum-starved HUVEC transduced with a control, wild type Raf1 (WT) or RAF1S259A (S259A) lentiviruses were stimulated with 50 ng/ml VEGF-A₁₆₄. Western blot demonstrates increased ERK1/2 phosphorylation increased in RAF1S259A cells. ERK activation was quantified by densitometry and is represented as ratio of pERK1/2 versus total ERK1/2. Immunofluorescence staining showing higher P-ERK1/2 in β-GAL positive endothelial cells of E12.5 S259A embryos. jv, jugular vein. FIGS. 10B-10E are graphs showing HUVEC transduced with control, wild type Raf1 or RAF1S259A lentiviruses treated with DMSO, MEK inhibitor U0126 (10 μM) or PI3K inhibitor LY294002 (10 μM) for 24 hours. Sox18 (FIG. 10B), Prox1 (Figure), Vegfr3 (FIG. 10D) and Lyve1 (10E) expression was assessed by qPCR. FIGS. 10E-10H are graphs showing HUVEC transduced with GFP or RAF1S259A adenoviruses treated with DMSO, Rapamycin (10 nM), AKT inhibitor VIII (1 μM) and LY294002 (10 μM) for 24 hours. Sox18 (FIG. 10F), Prox1 (FIG. 10G) and Coup-TFII (FIG. 10H) expression assessed by qPCR. Data are Mean±SEM (n=3).*, p<0.05; **, p<0.01.

FIGS. 11A-11J are graphs showing excessive ERK activation is the basis of lymphatic abnormalities in Noonan syndrome. HUVEC (FIGS. 11A-11D) and HDLEC (FIGS. 11E-11H) were transduced with adenovirus expressing lacZ, cytosolic localized constitutive active ERK (ME) or nuclear localized constitutive active ERK (LA). Sox18 (FIGS. 11A and 11E), Prox1 (FIGS. 11B and 11F), Vegfr3 (FIGS. 11C and 11G) and Lyve1 (FIGS. 11D and 11H) expression were assessed by qPCR. Data are Mean±SEM (n=3). FIG. 11I is a graph of serum starved HDLEC stimulated with 100 ng/ml VEGF-C for an indicated time in the presence of 10126 (10 μM) or an equal volume of DMSO. Sox18 expression was assessed by qPCR. Data are Mean±SEM (n=3). FIG. 11J is a graph of lymphatic vessel diameter calculated by whole mount staining of skin vasculature of E14.5 embryos treated with MEK inhibitor U0126 or DMSO. Statistical significance was determined by two-tailed t-test (Control, n=5 embryos; S259A, n=3 embryos).

FIGS. 12A and 12B are proposed models for molecular basis of lymphangiectasia. FIG. 12A is a proposed scheme of ERK-dependent regulation of PROX1 expression. ERK signaling induces lymphatic endothelial cell differentiation by controlling SOX18 expression. FIG. 12B is a proposed model of molecular basis of lymphangiectasia formation (modified from Francois et al (2008). Under normal condition, Sox18 is only transiently expressed in veins, which in turn initiates LEC specification by inducing Prox1. The PROX1 positive LECs then delaminate from veins and eventually form lymph sacs. In the setting of excessive ERK activation, there is an increased and persistent expression of Sox18 and Prox1 in veins, resulting in continuous induction of lymphatic fate in venous EC and excessive transition of these lymphatic EC into forming lymph sacs and vessels leading to lymphangiectasia.

### DETAILED DESCRIPTION OF THE INVENTION

### I. Definitions

As used herein, “Molecules that increase the bioavailability of non-phosphorylated RAF1” refers to ref to RAF1 mutants or nuclei acids encoding RAF1 mutants which cannot be phosphorylated by RAK, but retain kinase activity.

As used herein, “Molecules that decrease bioavailability of RAK 1” refers to molecules that decrease RAK1 protein levels or prevent interfere with RAK1-RAF1 binding.

As used herein, “Multiplicity of infection” (MOI) generally refers to the number of virions that are added per cell during infection.

As used herein, “operably linked” means incorporated into a genetic construct so that expression control sequences effectively control expression of a coding sequence of interest.

As used herein, “Small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

The term “protein” “polypeptide” or “peptide” refers to a natural or synthetic molecule comprising two or more amino acids linked by the carboxyl group of one amino acid to the alpha amino group of another.

The term “residue” as used herein refers to an amino acid that is incorporated into a protein. The amino acid may be a naturally occurring amino acid and, unless otherwise limited, may encompass known analogs of natural amino acids that can function in a similar manner as naturally occurring amino, acids.

The term “polynucleotide” or “nucleic acid sequence” refers to a natural or synthetic molecule comprising two or more nucleotides linked by a phosphate group at the 3′ position of one nucleotide to the 5′ end of another nucleotide. The polynucleotide is not limited by length, and thus the polynucleotide can include deoxyribonucleic acid (DNA) or ribonucleic acid (RNA).

The term “gene” refers to a polynucleotide that encodes a protein or functional RNA molecule.

The term “vector” or “construct” refers to a polynucleotide capable of transporting into a cell another polynucleotide to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of gene to a transcriptional control element refers to the physical and functional relationship between the gene and promoter such that the transcription of the gene is initiated from the promoter by an RNA polymerase that specifically recognizes, binds to and transcribes the DNA.

The terms “transformation” and “transfection” refer to the introduction of a polynucleotide, e.g., an expression vector, into a recipient cell including introduction of a polynucleotide to the chromosomal DNA of the cell.

The term “variant” refers to an amino acid or nucleic acid sequence having conservative substitutions, non-conservative substitutions (i.e. a degenerate variant), substitutions within the wobble position of a codon encoding an amino acid, amino acids added to the C-terminus of a peptide, or a peptide having 60%, 70%, 80%, 90%, 95%, 96%, 97%, 98%, or 99% sequence identity to an amino acid sequence.

The term “conservative variant” refers to a particular nucleic acid sequence that encodes identical or essentially identical amino acid sequences. Conservative substitution tables providing functionally similar amino acids are well known in the art. The following sets forth exemplary groups which contain natural amino acids that are “conservative substitutions” for one another. Conservative Substitution Groups 1: Alanine (A), Serine (S) Threonine (T); 2: Aspartic acid (D), Glutamic acid (E); 3: Asparagine (N), Glutamine (Q); 4: Arginine (R), Lysine (K); 5: Isoleucine (I) Leucine (L), Methionine (M), Valine (V); and 6: Phenylalanine (F), Tyrosine (Y), Tryptophan (W).

The term “percent (%) sequence identity” or “homology” refers to the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “translation system” refers to the components necessary to incorporate an amino acid into a growing polypeptide chain (protein). Components of a translation system generally include amino acids, ribosomes, tRNAs, synthetases, and mRNA.

The term “transgenic organism” refers to any organism, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. Suitable transgenic organisms include, but are not limited to, bacteria, cyanobacteria, fungi, plants and animals. The nucleic acids described herein can be introduced into the host by methods known in the art, for example infection, transfection, transformation or transconjugation.

The term “eukaryote” or “eukaryotic” refers to organisms or cells or tissues derived from these organisms belonging to the phylogenetic domain Eukarya such as animals (e.g., mammals, insects, reptiles, and birds), ciliates, plants (e.g., monocots, dicots, and algae), fungi, yeasts, flagellates, microsporidia, and protists.

The term “prokaryote” or “prokaryotic” refers to organisms including, but not limited to, organisms of the Eubacteria phylogenetic domain, such as Escherichia coli, Thermus thermophilus, and Bacillus stearothermophilus, or organisms of the Archaea phylogenetic domain such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, and Aeuropyrum pernix.

Various types of mutagenesis can be used to modify a nucleic acid. They include, but are not limited to, site-directed, random point mutagenesis, homologous recombination (DNA shuffling), mutagenesis using uracil containing templates, oligonucleotide-directed mutagenesis, phosphorothioate-modified DNA mutagenesis, and mutagenesis using methods such as gapped duplex DNA. Additional suitable methods include point mismatch repair, mutagenesis using repair-deficient host strains, restriction-selection and restriction-purification, deletion mutagenesis, mutagenesis by total gene synthesis and double-strand break repair.

Deletion variants are characterized by the removal of one or more nucleotides or amino acid residues from the nucleic acid or protein sequence. Deletions or substitutions of cysteine or other labile residues may be desirable, for example in increasing the oxidative stability or selecting the preferred disulfide bond arrangement. Deletions or substitutions of potential proteolysis sites, e.g. Arg Arg, are accomplished, for example, by deleting one of the basic residues or substituting one by glutaminyl or histidyl residues. Variants ordinarily are prepared by site specific mutagenesis of nucleotides in the DNA encoding the protein, thereby producing DNA encoding the variant, and thereafter expressing the DNA in recombinant cell culture. Variant fragments may also be prepared by in vitro synthesis. The variants typically exhibit the same qualitative biological activity as the naturally-occurring analogue.

Substitutional variants are those in which at least one residue sequence has been removed and a different residue inserted in its place. Owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, conservative amino acid substitutions are also readily identified. Such conservative variations are a feature of each disclosed sequence. The substitutions which in general are expected to produce the greatest changes in protein properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

While the site for introducing a nucleotide or amino acid sequence variation is predetermined, the mutation per se need not be predetermined. For example, in order to optimize the performance of a mutation at a given site, random mutagenesis may be conducted at the target codon or region and the expressed variants screened for the optimal combination of desired activity. Techniques for making substitution mutations at predetermined sites in DNA having a known sequence are well known.

Substitutions are typically of single residues; insertions usually will be on the order of about from 1 to 10 residues; and deletions will range about from 1 to 30 residues. Substitutions, deletion, insertions or any combination thereof may be combined to arrive at a final construct. The mutations that will be made in the DNA must not place the sequence out of reading frame and preferably will not create complementary regions that could produce secondary mRNA structure.

The precise percentage of similarity between sequences that is useful in establishing sequence identity varies with the nucleic acid and protein at issue, but as little as 25% sequence similarity is routinely used to establish sequence identity. Higher levels of sequence similarity, e.g., 30%, 40%, 50%, 60%, 70%, 80%, 90%, 95% or 99% or more can also be used to establish sequence identity. Methods for determining sequence similarity percentages (e.g., BLASTP and BLASTN using default parameters) are generally available.

Alignment of sequences for comparison can be conducted by many well-known methods in the art, for example, by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the Gibbs sampling method (Chatterji and Pachter, J Comput Biol. 12(6):599-608 (2005)), by PSI-BLAST-ISS (Margelevicius and Venclovas, BMC Bioinformatics 21; 6:185 (2005)), or by visual inspection. One algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Components of a translation system generally include amino acids, ribosomes, tRNAs, synthetases, and mRNA. In some embodiments, a cell-based (in vivo) expression system is used. In these embodiments, nucleic acids are delivered to cells under conditions suitable for translation and/or transcription. The cells can in some embodiments be prokaryotic, e.g., an E. coli cell, or eukaryotic, e.g., a yeast, mammalian, plant, or insect or cells thereof.

In some embodiments, a cell-free (in vitro) expression system is used. The most frequently used cell-free translation systems involve extracts containing all the macromolecular components (70S or 80S ribosomes, tRNAs, aminoacyl-tRNA synthetases, initiation, elongation and termination factors, etc.) required for translation of exogenous RNA. To ensure efficient translation, each extract is supplemented with amino acids, energy sources (ATP, GTP), energy regenerating systems (creatine phosphate and creatine phosphokinase for eukaryotic systems, and phosphoenol pyruvate and pyruvate kinase for the E. coli lysate), and other co-factors (Mg2+, K+, etc.).

Nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Suitable promoters are generally obtained from viral genomes (e.g., polyoma, Simian Virus 40 (SV40), adenovirus, retroviruses, hepatitis-B virus, and cytomegalovirus) or heterologous mammalian genes (e.g. beta actin promoter). Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers also often contain response elements that mediate the regulation of transcription. Many enhancer sequences are now known from mammalian genes (globin, elastase, albumin, α-fetoprotein and insulin). However, enhancers from a eukaryotic cell virus are preferably used for general expression. Suitable examples include the SV40 enhancer on the late side of the replication origin, the cytomegalovirus early promoter enhancer, the polyoma enhancer on the late side of the replication origin, and adenovirus enhancers.

In certain embodiments the promoter and/or enhancer region can act as a constitutive promoter and/or enhancer to maximize expression of the region of the transcription unit to be transcribed. In certain constructs the promoter and/or enhancer region is active in all eukaryotic cell types, even if it is only expressed in a particular type of cell at a particular time. A preferred promoter of this type is the CMV promoter. In other embodiments, the promoter and/or enhancer is tissue or cell specific.

In certain embodiments the promoter and/or enhancer region is inducible. Induction can occur, e.g., as the result of a physiologic response, a response to outside signals, or as the result of artificial manipulation. Such promoters are well known to those of skill in the art. For example, in some embodiments, the promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

There are a number of compositions and methods which can be used to deliver nucleic acids to cells, either in vitro or in vivo. These methods and compositions can largely be broken down into two classes: viral based delivery systems and non-viral based delivery systems. For example, nucleic acids can be delivered through a number of direct delivery systems such as electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are well known in the art and readily adaptable for use with the compositions and methods described herein.

Transfer vectors can be any nucleotide construction used to deliver genetic material into cells. In some embodiments the vectors are derived from either a virus or a retrovirus. Viral vectors include, for example, Adenovirus, Adeno-associated virus, Herpes virus, Vaccinia virus, Polio virus, AIDS virus, neuronal trophic virus, Sindbis and other RNA viruses, including these viruses with the HIV backbone.

Typically, viral vectors contain nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. The necessary functions of the removed early genes are typically supplied by cell lines which have been engineered to express the gene products of the early genes intrans.

Nucleic acids can also be delivered through electroporation, sonoporation, lipofection, or calcium phosphate precipitation. Lipofection involves the use liposomes, including cationic liposomes (e.g., DOTMA, DOPE, DC-cholesterol) and anionic liposomes, to delivery genetic material to a cell. Commercially available liposome preparations include LIPOFECTIN, LIPOFECTAMINE (GIBCO-BRL, Inc., Gaithersburg, Md.), SUPERFECT (Qiagen, Inc. Hilden, Germany), and TRANSFECTAM (Promega Biotec, Inc., Madison, Wis.).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can become integrated into the host genome. Techniques for integration of genetic material into a host genome are also known and include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

The vectors used to deliver the disclosed nucleic acids to cells can further include nucleic acid sequence encoding a marker product. This marker product is used to determine if the gene has been delivered to the cell and once delivered is being expressed. In some embodiments the marker is a detectable label. Exemplary labels include the E. coli lacZ gene, which encodes b-galactosidase, and green fluorescent protein (GFP).

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection.

### II. Compositions Stimulating Arteriogenesis or Lymphagenesis

ERK (Extracellular signal-regulated Kinase) is a major component of the mitogen-activated protein kinase (MAPK) cascades which form major intracellular signaling (Chang and Karin, 2001; Su and Karin, 1996). Each MAPK is activated by a MAPK kinase (MAPKK) that in turn is activated by a MAPKK kinase (MAP3K) (Chang and Karin, 2001). Correspondingly, ERK is activated by MEK1/2, which in turn is largely activated by a class of MAP3Ks, the RAF kinases (A-RAF, B-RAF, RAF1) (Chong et al., 2003). RAF1 is the most intensively studied RAF. It can be activated by VEGF and bFGF in endothelial cells and plays a vital role in vascular protection from apoptosis (Alavi et al., Science, 301:94-96 (2003)). Ablation of Raf1 (Mikula, et al., EMBO J., 20:1952-1962 (2001), Mek1 (Giroux et al., Curr Biol, 9:369-372 (1999)) and Erk2 (Yao et al., Proc. Nat. Acad. Sci. USA, 100:12759-12764 (2003)) all results in embryonic death due to vascular defects, suggesting that the RAF1-MEK-ERK pathway plays an essential role in vascular development.

Recent studies in zebra fish have shown that RAF-AKT crosstalk governs artery-vein specification during embryonic development Hong et al., Circ Res, 103:573-579 (2008)). Activation of ERK in zebra fish mutated in the gridlock gene, which encodes for a helix-loop-helix protein of the hairy and enhancer of split family of transcriptional repressors, is impaired (Hong et al., Curr Biol., 16:1366-1372 (2006)). The mutant fish have incompletely formed lateral aorta and lack circulation in the posterior trunk and tail (Weinstein et al., Nat Med, 1:1143-1147 (1995); Zhong et al., Science, 287:1820-1824 (2000)). Partial inhibition of PI3-kinsase activity with a PI3K inhibitor, GS4898, or known PI3K inhibitors, LY294002 and wortmannin, led to a recovery of ERK activation and a virtually complete rescue of the distal arterial circulation (Hong et al., Curr Biol., 16:1366-1372 (2006)). Mosaic transgenic expression of AKT showed that dominant negative AKT was preferentially localized in dorsal aorta, whereas expression of constitutively active AKT was observed mainly in the posterior cardinal vein (Hong et al., Curr Biol., 16:1366-1372 (2006)). Importantly, during zebra fish development, activated ERK is localized precisely to dorsal angioblasts that will develop into aortic endothelial cells but not ventral angioblasts destined to become venous endothelial cells (Hong, et al., Curr Biol., 16:1366-1372 (2006)). Analogous aortic-specific ERK activation has also been observed in mouse embryos (Corson et al., Development, 130:4527-4537 (2003)). Taken together, these findings suggest that RAF-MEK-ERK signaling is required for the arterial fate, whereas PI3K-AKT signaling has an opposing effect by inhibiting the RAF-MEK-ERK pathway.

Several studies have recently implicated ERK in a potential role in arteriogenesis. High levels of phosphorylated ERK are found in growing collateral arteries (Eitenmuller et al., Circulation Res., 99:656-662 (2006)) and sheer stress, a major factor that is thought to trigger arteriogenesis (Simons, Circulation, 111:1556-1566 (2005)), is capable of inducing ERK activation (Jalali et al., Arteriosclerosis, Thrombosis, and Vascular Biology, 18:227-234 (1998); Sumpio et al., J. Biol. Chem., 280:11185-11191 (2005)). Furthermore, ERK activity is profoundly reduced in the vasculature of both zebrafish (gridlock) (Hong et al., Curr Biol., 16:1366-1372 (2006)) and mouse (Synectin) (Chittenden et al., Dev Cell, 10:783-795 (2006)) mutants which were demonstrated to be defective in arteriogenesis.

ERK activation is markedly reduced in synectin^{-/-} arterial endothelial cells in response to VEGF-A. An increase in ERK activity either by partial inhibition of PI3-kinase or by direct introduction of a constitutively active MEK-ERK construct rescued the ability of synectin^{-/-} arterial endothelial cells to migrate and to form branching vascular structures in vitro and restored arterial morphogenesis in synectin knockdown zebrafish. In addition, the same manipulations were effective in restoring arteriogenesis in adult synectin^{-/-} mice and in LDL-R^{-/-}/ApoB48-deficient mice maintained on high fat diet. Together, given the fact that ERK activity is tightly controlled by the RAF-AKT crosstalk, RAF1-AKT crosstalk must play an important role in arteriogenesis.

Extracellular signals often result in simultaneous activation of both the RAF-MEK-ERK and PI3K-AKT pathways. However, these two pathways exert opposing effects on a variety of biological events such as muscle cell hypertrophy, and vascular smooth muscle cell growth and differentiation (Moelling et al., J Biol Chem, 277:31099-31106 (2002); Reusch et al., J Biol Chem, 276:33630-33637 (2001); Rommel et al., Science, 286:1738-1741 (1999)). Besides acting in parallel, these two pathways also cross talk. In vitro, RAF1 activity was shown to be inhibited by AKT (Rommel et al., 286:1738-1741 (1999); Zimmermann and Moelling, Science, 286:1741-1744 (1999)). RAF1 can be phosphorylated by AKT at Ser259 in the regulatory domain, which in turn recruits the 14-3-3 protein, a negative regulator of RAF1. Based on these findings, it has been established that the RAF-MEK-ERK and the PI3K-AKT pathways crosstalk on the level of RAF and AKT.

The Notch signaling is another pathway implicated in both artery-vein specification and arteriogenesis. In mammals, four notch family receptors (Notch1-4) and five Notch ligands: Jagged (Jag 1, 2), Delta-like ligands (Dll1, 3, 4) have been discovered (Swift and Weinstein, Circ Res, 104:576-588 (2009)). Upon ligand binding, three proteolytic cleavages occurs and eventually leads to a subsequent release of the Notch intracellular domain (NICD) catalyzed by the γ-secretase in the final step. NICD is then translocated to the nucleus and regulates gene expression such as Hes and Hey (Fortin, Dev Cell, 16:633-647 (2009); Gridley, Development, 134:2709-2718 (2007)). NOTCH1 and NOTCH4 as well as the NOTCH ligands JAG1, JAG2, DLL1 and DLL4 are selectively expressed in arterial endothelial cells (Swift and Weinstein, Circ Res., 104:576-588 (2009)). To date, a collection of studies have suggested that Notch signaling is required for both embryonic and postnatal arterial development (Benedito and Duarte, Gene Expr Patterns, 5:750-755 (2005); Benedito et al., BMC Dev Biol, 8:117 (2008); Duarte et al., Genes Dev, 18:2474-2478 (2004); Hofmann, et al., Gene Expr Patterns, 7:461-470 (2007); Krebs et al., Genes Dev, 18:2469-2473 (2004); Krebs et al., Genes Dev, 14:1343-1352 (2000); Leslie et al., Development, 134:839-844 (2007); Shutter et al., Genes Dev, 14:1313-1318 (2000); Sorensen et al., Blood, 113:5680-5688 (2009); Takeshita et al., Circ Res, 100:70-78 (2007); Trindade et al., Blood, 112:1720-1729 (2008)).

Of the five NOTCH ligands, DLL4 is of particular interest due to its specific role in regulating tip cell formation (Hellstrom et al., Nature, 445:776-780 (2007); Siekmann and Lawson, Nature, 445:781-784 (2007); Suchting et al., Proc Natl Acad Sci USA, 104:3225-3230, 2007) and embryonic artery development (Duarte et al., Genes Dev, 18:2474-2478 (2004); Gale et al., Proc Natl Acad Sci USA, 101:15949-15954 (2004)). DLL4 acts in a dose-dependent manner (Duarte et al., Genes Dev, 18:2474-2478 (2004); Gale et al., Proc Natl Acad Sci USA, 101:15949-15954 (2004), indicating that precise control of DLL4 expression is of vital importance for normal vascular development. It has been shown that DLL4 and Hey2 expression are regulated by PI3K-AKT pathway (Hayashi and Kume, PLoS One, 3:e2401 (2008); Liu et al., Mol Cell Biol, 23:14-25 (2003)), and possibly by RAF-MEK-ERK pathway as well (Hong et al., Circ Res, 103:573-579 (2008)). However, the studies have been controversial because important differences have been uncovered from in vitro mammalian cell studies versus in vivo zebrafish studies regarding interactions between VEGF, Notch signaling and ERK signaling. In zebrafish, ERK activation is believed to regulate arterial marker expression such as DLL4 via FoxC1/2 (Hong et al., Circ Res, 103:573-579 (2008)), while in mammalian arterial endothelial cells, both VEGF and FoxC1/2-induced DLL4 expression is dependent of the PI3K-AKT pathway but independent of ERK (Hayashi and Kume, PLoS One, 3:e2401 (2008); Liu et al., Mol Cell Biol, 23:14-25 (2003)). So far, it is still largely unknown how DLL4-Notch signaling is regulated both in vitro and in vivo. Molecular mechanisms controlling arterial growth are not understood and, as a result, there have been no drugs developed to stimulate it.

Previous studies have shown that downregulation of PI3K activity or introduction of a constitutively active ERK1/2 construct restored angiogenesis and arteriogenesis in synectin^{-/-} mice and synectin KD zebrafish. Ren, et al., J. Clin Invest., 120(4):1217-28 (2010). However, PI3K kinase inhibitors are toxic, and while suppression of Pi3K/Akt signaling will activate ERK, this occurs at the expense of suppressing Akt1 and eNOS activation.

Molecular mechanisms controlling arterial growth are not understood and, as a result, there have been no drugs develop to stimulate it. A single amino acid mutation in Raf1 (Ser259 to Ala259) renders RF resistant to inhibition by PI3K/Akt signaling. This results in simultaneous activation of both Raf/ERK and PI3K/Akt signaling pathway, something that does not normally happen, and a full re-expression of the entire embryonic arteriogenic program. Importantly, RAF1S259A acts as a dominant-positive in normal cells. The compositions provided herein prevent RAF1-AKT crosstalk and allow simultaneous activation of RAF/ERK and PI3K/Akt signaling pathway, resulting in a full re-expression of the entire embryonic arteriogenic program.

The compositions for increasing arteriogenesis include molecules that increase the bioavailability of non-phosphorylated RAF1, molecules that decrease the bioavailability of RAK1, and constitutively active ERK for local or directed delivery, alone or in combination. The compositions also can also include other pharmaceutically active agents.

A. Molecules Increasing Raf1 (Ser259 to Ala259) Bioavailability

1. Proteins and Polypeptides

In some embodiments, the composition for increasing bioavailability of non-phosphorylated RAF1 includes a RAF1 mutant which cannot be phosphorylated by RAK1 but retains the RAF1 kinase activity. A preferred RAF1 mutant is the RAF1 Ser259 to Ala259 mutant i.e., a RAF1 S259A protein or polypeptide fragment thereof or a variant thereof retaining its activity. Variants of RAF1S259A include polypeptide sequences which may have minor base pair changes which may result in variation (conservative substitution) in the amino acid sequence encoded. Such conservative substitutions are not expected to substantially alter the biologic activity of the gene product. A conservative substitution or modification of one or more amino acids includes substitutions such that the tertiary configuration of the protein is substantially unchanged. Conservative substitutions include substitutions of amino acids having substantially the same charge, size, hydrophobicity, and/or aromaticity as the amino acid replaced. Such substitutions, known to those of ordinary skill in the art, include glycine-alanine-valine; isoleucine-leucine; tryptophan-tyrosine; aspartic acid-glutamic acid; arginine-lysine; asparagine-glutamine; and serine-threonine. The importance of the effect of amino acid substitutions on the local and global folding of a polypeptide is generally understood in the art (see, e.g., Bordo & Argos, J. Mol. Biol., 217:721-729 (1991); Jones, J. Theor. Biol, 50:167-183 (1975); Hoop and Woods, Proc Natl Acad Sci USA 78:3824 (1981) and Sweet & Eisenberg, J Mol Biol 171(4): 479-88(1983)). Conservative substitutions by functionally equivalent residues can be performed based on charge and topological structure conservation. One of ordinary skill in the art can make such substitutions chemically, or alternatively, produce a nucleic acid molecule which encodes a conservative variant of RAF1S259A by using methods readily available to one of ordinary skill in the art, e.g., site-directed mutagenesis of an isolated nucleic acid molecule followed by expression of the molecule, or by chemical synthesis.

The S259A protein or polypeptide can be incorporated into a pharmaceutically acceptable carrier for intravenous or parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN 20, TWEEN 80, also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

In a preferred embodiment, discussed below, these formulations are designed for local delivery, and more preferably, release over a limited time frame, thereby increasing specificity and decreasing risk of adverse side effects.

2. Nucleic Acid Molecules

In other embodiments the composition for increasing bioavailability of non-phosphorylated RAF1 comprises an isolated nucleic acid molecule encoding RAF1S259A, polypeptide fragment thereof or a variant thereof retaining activity, as described above.

Compositions and methods for increasing stability of nucleic acid half-life and nuclease resistance are known in the art, and can include one or more modifications or substitutions to the nucleobases, sugars, or linkages of the polynucleotide. For example, the polynucleotide can be custom synthesized to contain properties that are tailored to fit a desired use. Common modifications include, but are not limited to use of locked nucleic acids (LNAs), unlocked nucleic acids (DNAs), morpholinos, peptide nucleic acids (PNA), phosphorothioate linkages, phosphonoacetate, linkages, propyne analogs, 2′-O-methyl RNA, 5-Me-dC, 2′-5′ linked phosphodiester linage, chimeric Linkages (mixed phosphorothioate and phosphodiester linkages and modifications), conjugation with lipid and peptides, and combinations thereof.

In some embodiment, the polynucleotide includes internucleotide linkage modifications such as phosphate analogs having achiral and uncharged intersubunit linkages, or uncharged morpholino-based polymers having achiral intersubunit linkages. Some internucleotide linkage analogs include morpholidate, acetal, and polyamide-linked heterocycles. A locked nucleic acid (LNA), often referred to as inaccessible RNA, is a modified RNA nucleotide where the ribose moiety is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The locked ribose conformation enhances base stacking and backbone pre-organization. Oligonucleotides incorporating LNAs have increased thermal stability and improved discriminative power with respect to their nucleic acid targets. Commercial nucleic acid synthesizers and standard phosphoramidite chemistry are used to make LNAs. Other backbone and linkage modifications include, but are not limited to, phosphorothioates, peptide nucleic acids, tricyclo-DNA, decoy oligonucleotide, ribozymes, spiegelmers (containing L nucleic acids, an apatamer with high binding affinity), or CpG oligomers.

Phosphorothioates (or S-oligos) are a variant of normal DNA in which one of the nonbridging oxygens is replaced by a sulfur. The sulfurization of the internucleotide bond dramatically reduces the action of endonucleases and exonucleases including 5′ to 3′ and 3′ to 5′ DNA POL 1 exonuclease, nucleases S1 and P1, RNases, serum nucleases and snake venom phosphodiesterase. In addition, the potential for crossing the lipid bilayer increases. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1,2-bensodithiol-3-one 1,1-dioxide (BDTD). The latter methods avoid the problem of elemental sulfur's insolubility in most organic solvents and the toxicity of carbon disulfide. The TETD and BDTD methods also yield higher purity phosphorothioates.

Peptide nucleic acids (PNA) are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that is similar to oligonucleotides, but are achiral and neutrally charged molecules. Peptide nucleic acids are typically comprised of peptide nucleic acid monomers. The heterocyclic bases can be any of the standard bases (uracil, thymine, cytosine, adenine and guanine) or any of the modified heterocyclic bases described below. A PNA can also have one or more peptide or amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), and the like. Methods for the chemical assembly of PNAs are well known.

In some embodiments, the polynucleotide includes one or more chemically-modified heterocyclic bases including, but not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-b-D-ribofuranosyl)pyridine (2-aminopyridine), and various pyrrolo- and pyrazolopyrimidine derivatives, 4-acetylcytosine, 8-hydroxy-N-6-methyladenosine, aziridinylcytosine, 5-(carboxyhydroxylmethyl) uracil, 5-bromouracil, 5-carboxymethylaminomethyl-2-thiouracil, 5-carboxymethylaminomethyluracil, dihydrouracil, N6-isopentenyladenine, 1-methyladenine, 1-methylpseudouracil, 1-methyl guanine, 1-methylinosine, 2,2-dimethylguanine, 2-methyladenine, 2-methylguanine, 3-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, 2,6-diaminopurine, and 2′-modified analogs such as, but not limited to O-methyl, amino-, and fluoro-modified analogs.

In some embodiments the polynucleotide include one or more sugar moiety modifications, including, but are not limited to, 2′-O-aminoethoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME), and 2′-O—(N-(methyl)acetamido) (2′-OMA).

The nucleic acid molecule maybe incorporated in any carrier suitable for gene delivery. Suitable gene delivery systems may include liposomes, receptor-mediated delivery systems, naked DNA, and viral vectors such as herpes viruses, retroviruses, adenoviruses, and adeno-associated viruses, among others. Delivery of nucleic acid molecules to a specific site in the body for gene therapy may also be accomplished using a biolistic delivery system, such as that described by Williams (Proc. Natl. Acad. Sci. USA 88 (1991), 2726-2729 (1991). Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a variant costimulatory polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. RAF1S259A-encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein. Nucleic acids encoding polypeptides can be administered to subjects in need thereof. Nucleic delivery involves introduction of “foreign” nucleic acids into a cell and ultimately, into a live animal. Compositions and methods for delivering nucleic acids to a subject are known in the art (see Understanding Gene Therapy, Lemoine, N. R., ed., BIOS Scientific Publishers, Oxford, 2008).

In some embodiments, DNA encoding the nucleic acid is incorporated into an expression vector. Methods to construct expression vectors containing genetic sequences and appropriate transcriptional and translational control elements are well known in the art. These methods include in vitro recombinant DNA techniques, synthetic techniques, and in vivo genetic recombination. Vectors include, but are not limited to, plasmids, viral nucleic acids, viruses, phage nucleic acids, phages, cosmids, and artificial chromosomes.

In some embodiments, the vector is derived from either a virus or a retrovirus. Viral vectors include adenovirus, adeno-associated virus, herpes virus, vaccinia virus, polio virus, HIV virus, neuronal trophic virus, Sindbis and other RNA viruses. Also useful are any viral families which share the properties of these viruses which make them suitable for use as vectors. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. The necessary functions of the removed early genes are typically supplied by cell lines which have been engineered to express the gene products of the early genes in trans.

Expression vectors generally contain regulatory sequences, necessary elements for the translation and/or transcription of the inserted coding sequence. For example, the coding sequence is preferably operably linked to a promoter and/or enhancer to control the expression of the desired gene product.

One skilled in the art will recognize that the selection of the promoter to express the gene of interest will depend on the vector, the nucleic acid cassette, the cell type to be targeted, and the desired biological effect. One skilled in the art will also recognize that in the selection of a promoter the parameters can include: achieving sufficiently high levels of gene expression to achieve a physiological effect; maintaining a critical level of gene expression; achieving temporal regulation of gene expression; achieving cell type specific expression; achieving pharmacological, endocrine, paracrine, or autocrine regulation of gene expression; and preventing inappropriate or undesirable levels of expression. Any given set of selection requirements will depend on the conditions but can be readily determined once the specific requirements are determined.

Promoters can generally be divided into constitutive promoters, tissue-specific or development-stage-specific promoters, inducible promoters, and synthetic promoters. Constitutive promoters direct expression in virtually all tissues and are largely, if not entirely, independent of environmental and developmental factors. As their expression is normally not conditioned by endogenous factors, constitutive promoters are usually active across species and even across kingdoms. A preferred promoter of this type is the CMV promoter (650 bases).

Tissue-specific or development-stage-specific promoters direct the expression of a gene in specific tissue(s) or at certain stages of development.

The performance of inducible promoters is not conditioned to endogenous factors but to environmental conditions and external stimuli that can be artificially controlled. Within this group, there are promoters modulated by abiotic factors such as light, oxygen levels, heat, cold, and wounding. Since some of these factors are difficult to control outside an experimental setting, promoters that respond to chemical compounds, not found naturally in the organism of interest, are of particular interest.

An enhancer is a sequence of DNA that functions at no fixed distance from the transcription start site and can be either 5′ or 3′ to the transcription unit. Furthermore, enhancers can be within the coding sequence itself. They are usually between 10 and 300 bp in length, and they function in cis. Enhancers function to increase transcription from nearby promoters. Enhancers also often contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Expression vectors used in eukaryotic host cells may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

The polynucleotides can be formulated for administration to a subject. In some embodiments, the disclosed formulation contains one or more polynucleotides (e.g., 0.1 to 90% by weight) mixed with physiologically acceptable excipients and/or additives. Preferred physiologically acceptable carrier excipients for injection are water, buffered water, normal saline, 0.4% saline, or 0.3% glycine. Suitable pharmaceutical excipients include stabilizers, antioxidants, osmolality adjusting agents, buffers, and pH adjusting agents.

Tremendous strides have been made in developing strategies for DNA/RNA delivery into cells that can protect it from degradation and facilitate targeted cellular uptake. The choice of strategy is determined by the DNA-based therapeutic of interest and its final clinical outcome. Nuclease degradation of oligonucleotides and other shorter DNA therapeutics can be circumvented by chemical derivatization of the backbone and/or by the protection and stability offered by DNA delivery systems.

In a preferred embodiment, the polynucleotide formulation is suitable for delivery to a cell in vivo, e.g., to a cell in an organism. In another embodiment, the polynucleotide formulation is suitable for delivery to a cell in vitro, e.g., to a cell in a cell line in culture or a suspension. The polynucleotide formulation can include a ligand that is selected to improve stability, distribution or cellular uptake of the agent. For example, the ligand can be a lipophilic moiety, e.g., cholesterol, which enhances entry of the polynucleotide into a cell.

Rapid escape and protection from the endosomal degradation can been achieved by the inclusion of fusogenic lipids such as 1,2-dioleoyl-sn-glycero-3-phosphoethanolamine (DOPE) in pH-sensitive and cationic liposome delivery systems. DOPE is a helper lipid capable of disrupting the endosomal membrane upon endosomal acidification by the formation of lipid hexagonal phases. Endosomal membrane disruption can release the DNA-based therapeutic and its delivery system into the cytoplasm. Lysosomatropic agents such as monensin and chloroquine, which raise the endosomal pH, block acidification, and thus inhibit lysozyme activity, have also been used to facilitate endosomal release of DNA. Endosomal degradation of DNA-based therapeutics can also be circumvented by the incorporation of viral peptides such as hemagglutinin HA2 and those derived from adenoviruses in their delivery systems. Hemagglutinin HA2 undergoes conformational transition and leads to the destruction of the endosome, thereby facilitating the release of the DNA-based therapeutic. Enhanced rapid endosomal escape and enhanced transfection have also been achieved using fusogenic peptides such as poly(L-lysine) (PLL) and cationic polymers such as polyethylenimine (PEI) and dendrimers.

Polymer-DNA complexes, also known as polyplexes, can also be used to deliver DNA into cells. The polyplexes involve an electrostatic interaction between cationic polymers and anionic DNA. The cationic polyplex can then interact with the negatively charged cell surface to improve DNA uptake. Polymeric matrices with varying properties can be designed by choosing an appropriate distribution of different molecular weights and degree of cross-linking of the polymer, and/or by the incorporation of targeting ligands. Commonly used polymers include polyethylenimine, polylysine, chitosans, and dendrimers. Agents such as folates, transferrin, antibodies, or sugars such as galactose and mannose can be incorporated for tissue targeting.

The disclosed polynucleotides can be incorporated into a delivery vehicle, e.g., a liposome or a particle (e.g., a microparticle). Liposomes can be used as DNA drug delivery systems either by entrapping the polynucleotides inside the aqueous core or complexing them to the phospholipid lamellae. Since the phospholipid composition in the liposome bilayers can be varied, liposomal delivery systems can be easily engineered to yield a desired size, surface charge, composition, and morphology. A variety of cationic, anionic, synthetically modified lipids, and combinations thereof have been used to deliver a wide range of DNA-based therapeutics.

Cationic liposomal formulations generally contain mixtures of cationic and zwitterionic lipids. Cationic lipids commonly used are 1,2-dioleoyl-3-trimethylammonium propane (DOTAP), N-[1-(2,3-dioleyloxy)propyl]-N,N,Ntrimethylammonium chloride (DOTMA), 2,3-dioleoyloxyN-[2-(spermine carboxamido)ethyl]-N,N-dimethyl-1-propanaminium (DOSPA), dioctadecyl amido glycil spermine (DOGS), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA) and 1,2-dilinolenyloxy-N,N-dimethyl-3-aminopropane (DLenDMA), and 3,[N—(N1,N-dimethylethylenediamine)-carbamoyl]cholesterol (DC-chol). Commonly used zwitterionic lipids, also known as helper lipids, are DOPE and cholesterol. The cationic lipids in the liposomal formulation serve as a DNA complexation and DNA condensation agent during the formation of the lipoplex. The positive charge also helps in cellular association. The zwitterionic lipids help in membrane perturbation and fusion. Proprietary formulations of cationic lipids such as Lipofectamine (Invitrogen Carlsbad, Calif.), Effectene (Qiagen, Valencia, Calif.), and Tranfectam (Promega, Madison, Wis.) are commercially available.

In some embodiments, the disclosed polynucleotide formulation can include an aminoglycoside ligand, which can cause the polynucleotides to have improved hybridization properties or improved sequence specificity. Exemplary aminoglycosides include glycosylated polylysine; galactosylated polylysine; neomycin B; tobramycin; kanamycin A; and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. In some embodiments the guanidine analog (the guanidinoglycoside) of an aminoglycoside ligand is tethered to an oligonucleotide agent. In a guanidinoglycoside, the amine group on the amino acid is exchanged for a guanidine group. Attachment of a guanidine analog can enhance cell permeability of an oligonucleotide agent.

The disclosed polynucleotides can be formulated in combination with one or more additional agents, e.g., another therapeutic agent or an agent that stabilizes the polynucleotides. In some embodiments, the disclosed compositions contain chelators, salts, and RNAse inhibitors.

In some embodiment, the disclosed formulations contain a combination of polynucleotides. For example, in some embodiments, miR-520d and miR-224 polynucleotides are combined in the same formulation.

In some embodiment, the disclosed formulations contain one or more therapeutic or diagnostic compounds.

b. Molecules for Reducing AKT1 Bioavailability

The bioavailability of AKT1 can be reduced in one embodiment using RNA interference, whereby double-stranded RNA (d5RNA, also referred to herein as siRNAs or ds siRNAs, for double-stranded small interfering RNAs) induces the sequence-specific degradation of targeted mRNA in cells (Hutvagner and Zamore, Curr. Opin. Genet. Dev.: 12, 225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), or by micro-RNAs (miRNA), functional small-hairpin RNA (shRNA), or other dsRNAs which can be expressed in vivo using DNA templates with RNA polymerase III promoters (Zeng et al., Mol. Cell 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Sui et al., Proc. Natl. Acad. Sci. USA 99(6):5515-5520 (2002)).

SiRNA specific for AKT1 is commercially available. In addition, there are a number of companies that will generate interfering RNAs for a specific gene. Thermo Electron Corporation (Waltham, Mass.) has launched a custom synthesis service for synthetic short interfering RNA (siRNA). Each strand is composed of 18-20 RNA bases and two DNA bases overhang on the 3′ terminus. Dharmacon, Inc. (Lafayette, Colo.) provides siRNA duplexes using the 2′-ACE RNA synthesis technology. Qiagen (Valencia, Calif.) uses TOM-chemistry to offer siRNA with high individual coupling yields (Li, et al., Nat. Med., 11(9):944-951 (2005).

In other embodiments the AKT1 inhibitory molecule is an antisense oligonucleotide. An “antisense” nucleic acid sequence (antisense oligonucleotide) can include a nucleotide sequence that is complementary to a “sense” nucleic acid encoding a protein, e.g., complementary to the coding strand of a double-stranded cDNA molecule or complementary to the AKT1 mRNA. Antisense nucleic acid sequences and delivery methods are well known in the art (Goodchild, Curr. Opin. Mol. Ther., 6(2):120-128 (2004); Clawson, et al., Gene Ther., 11(17):1331-1341 (2004)), which are incorporated herein by reference in their entirety. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In other embodiments the AKT1 inhibitory molecule is a ribozyme specific for AKT1. Ribozymes are a type of RNA that can be engineered to enzymatically cleave and inactivate other RNA targets in a specific, sequence-dependent fashion. Ribozymes and methods for their delivery are well known in the art (Hendry, et al., BMC Chem. Biol., 4(1):1 (2004); Grassi, et al., Curr. Pharm. Biotechnol., 5(4):369-386 (2004); Bagheri, et al., Curr. Mol. Med., 4(5):489-506 (2004); Kashani-Sabet M., Expert Opin. Biol. Ther., 4(11):1749-1755 (2004), each of which are incorporated herein by reference in its entirety. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art.

In still other embodiments, the AKT1 inhibitory molecule can be an antibody specific for AKT1, preferably, a monoclonal antibody. A monoclonal antibody composition is typically composed of antibodies produced by clones of a single cell called a hybridoma that secretes (produces) only one type of antibody molecule. The hybridoma cell is formed by fusing an antibody-producing cell and a myeloma or other self-perpetuating cell line. Such antibodies were first described by Kohler and Milstein, Nature, 1975, 256:495-497, the disclosure of which is herein incorporated by reference. An exemplary hybridoma technology is described by Niman et al., Proc. Natl. Acad. Sci. U.S.A., 1983, 80:4949-4953. Other methods of producing monoclonal antibodies, a hybridoma cell, or a hybridoma cell culture are also well known. See for example, Antibodies: A Laboratory Manual, Harlow et al., Cold Spring Harbor Laboratory, 1988; or the method of isolating monoclonal antibodies from an immunological repertoise as described by Sasatry, et al., Proc. Natl. Acad. Sci. USA, 1989, 86:5728-5732; and Huse et al., Science, 1981, 246:1275-1281.

c. Additional Pharmaceutical Agents

Pharmaceutical agents which may be administered in the compositions disclosed herein include, but are not limited to, small molecule drugs for example, small molecules that interfere with the Raf1/Akt interaction such as LY294002 and Rapamycin, oligonucleotides, peptides and proteins which can inhibit the negative remodeling response, stimulate angiogenesis or regeneration of cardiac tissue. Cell survival promoting factors can also be used to increase the survivability of implanted cells.

The agent is preferably an agent that would benefit a damaged blood vessel or an infarcted area for example, by creating new cells or new cell components or trigger a repair mechanism. Suitable agents include, but are not limited to, growth factors (e.g., vascular endothelial growth factor (VEGF), fibroblast growth factor (FGF), platelet-derived growth factor (PDGF), insulin-like growth factor (IGF), hepatocyte growth factor (HGF), basic fibroblast growth factor (bFGF), acidic fibroblast growth factor (aFGF), placental growth factor (PlGF), granulocyte colony-stimulating factor (G-CSF)), cellular components, proteins and cytokines.

Bioactive agent used to induce regression or slow progress of an atherosclerotic plaque can also be administered with the compositions disclosed herein. Examples include apolipoprotein A1 (Apo A1) or a mutant or mimic form of Apo A1, or a molecule mimicking the cholesterol transporting capacity of ApoA1.

Other drugs that could be used include HDL mimetics, for example, cyclodestrin; anti-inflammatory agents, for example, clobetasol, dexamethasone, prednisone, aspirin and cordisone; and anti-proliferative agents for example taxol, everolomus, sirolomus, doxorubicin.

d. Devices and Implants

In preferred embodiments, the compositions are incorporated on, in, or with implants. For example, these can be incorporated into a coating on a stent for treating stenosis or a vascular graft, so that in the event re-stenosis develops, the composition has been released into the surrounding tissue to promote peripheral vascularization so that there is no impairment in blood flow. Even in the event that the graft is successful, this could be useful in enhancing blood flow around the stenotic vasculature. In another embodiment, the coatings are used to enhance vascularization into a graft, promoting tissue growth as well as new blood vessel growth, such as in the case of an orthopedic prosthetic which may otherwise show poor interfacing with the adjacent bone and connective tissue.

Medical devices that may incorporate the formulations include sutures, stents, stent grafts, stent coatings, devices for temporary wound or tissue support, devices for soft or hard tissue repair, repair patches, tissue engineering scaffolds, retention membranes, anti-adhesion membranes tissue separation membranes, hernia repair devices, device coatings, cardiovascular patches, catheter balloons, vascular closure devices, slings, biocompatible coatings, rotator cuff repair devices, meniscus repair devices, adhesion barriers, guided tissue repair/regeneration devices, articular cartilage repair devices, nerve guides, tendon repair devices, intracardiac septal defect repair devices, including, but not limited to, atrial septal defect repair devices and PFO (patent foramen ovale) closure devices, left atrial appendage (LAA) closure devices, pericardial patches, bulking and filling agents, plastic surgery devices (including facial and breast cosmetic and reconstructive devices), vein valves, heart valves, bone marrow scaffolds, meniscus regeneration devices, ligament and tendon grafts, ocular cell implants, spinal fusion devices, imaging devices, skin substitutes, dural substitutes, bone graft substitutes, wound dressings, and hemostats.

A major limiting factor in many implants is that cells cannot migrate in and proliferate until after formation of new vasculature. By incorporating these compositions into the implants, such as a polymeric mesh or microparticles which are administered at the site of a graft, the rate of vascularization and proliferation of new tissue is enhanced.

Microparticles which release these factors may also be used in conjunction with tissue implants, especially in the case of reattachment of digits, skin grafts in burn patients, and other applications with a need for extensive vasculature but which may not be amenable to surgical attachment of blood vessels within the site. The microparticles can be injected or perfused into the site of implantation and/or the graft, prior to or at the time of implantation. This is a preferred method of administration into areas of ischemic damage, such as the heart.

Methods of making microparticles and nanoparticles and incorporation of therapeutic, prophylactic or diagnostic agents onto or in the microparticles are well known to those skilled in the art. These may be injected into or adjacent to the site where lymphagenesis or arteriogenesis is desired, or injected systemically, with targeting ligands, size selection or blood vessel into which the particles are injected (such as the hepatic or coronary artery) being used to direct the particles to the desired target site. Selection of the polymer composition and molecular weight used to form the particles, the size of the particles, the method of formation and the location of the agents within or on the particles are used to regulate the pharmacokinetics of release. Representative polymers include polycaprolactones; poly(orthoesters); polyanhydrides; poly(phosphazenes); poly(lactide-co-caprolactones); poly(glycolide-co-caprolactones); polycarbonates such as tyrosine polycarbonates; polyamides (including synthetic and natural polyamides), polypeptides, and poly(amino acids); polyesteramides; other biocompatible polyesters; poly(dioxanones); poly(alkylene alkylates); hydrophilic polyethers; polyurethanes; polyetheresters; polyacetals; polycyanoacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), polyvinyl alcohols, polyvinylpyrrolidone; derivativized celluloses such as alkyl celluloses (e.g., methyl cellulose), hydroxyalkyl celluloses (e.g., hydroxypropyl cellulose), cellulose ethers, cellulose esters, nitrocelluloses, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), as well as derivatives, copolymers, and blends thereof.

Other techniques can also be used to provide controlled or sustained release at a site thereof. For example, administration in a hydrogel or other polymeric depot may be utilized. Examples of preferred natural polymers include proteins such as albumin, collagen, gelatin and prolamines, for example, zein, and polysaccharides such as alginate, pectin, cellulose derivatives and polyhydroxyalkanoates, for example, poly-(3-hydroxybutyrate) and poly (4-hydroxybutyrate). While a variety of polymers may be used to form the polymeric matrix, generally, the resulting polymeric matrix will be a hydrogel. For example, the matrix can also be made of a gel-type polymers, such as alginate, produced through traditional ionic gelation techniques.

### III. Methods of Treatment

The compositions are used to stimulate arteriogenesis or growth of lymphatic vasculature in subjects in need thereof, for example, in patients with conditions associated with defective arterial development or arterial insufficiency such as advanced coronary, peripheral or cerebral artery diseases and ischemic cardiomyopathy, for example atherosclerosis and diabetes. Examples of such diseases include stroke, ischemic heart failure, critical limb ischemia, angina, chronic stable angina, claudication, and lymphatic circulation deficiency.

In a preferred embodiment, the RAF1 S259A polypeptide, nucleic acid molecules encoding the RAF1 S259A, or RAF1 inhibitory molecules are administered either alone or in combination, in pharmaceutically acceptable carrier or excipient for intracoronary, intramuscular, intraarterial, intravenous, intraperitoneal or subcutaneous administration.

In other embodiments, the method comprises administering to a subject in need thereof, a nucleic acid molecule encoding RAF1 S259A, a RAK1 inhibiting nucleic acid, or a variant thereof, alone or in combination.

In other embodiments, the method comprises (a) obtaining cells, from a subject; (b) introducing into the cells a nucleic acid molecule encoding and capable of expressing the RAF1 S259A in vivo; and (c) reintroducing the cells obtained in step (b) into a subject in need thereof. Nucleic acids, such as those described above, can be inserted into vectors for expression in cells.

Nucleic acids in vectors can be operably linked to one or more expression control sequences. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the RAF1 S259A polypeptides described herein.

In other embodiments, the composition is administered locally. In these embodiments, the composition can be coated on or incorporated into a vascular device, such as a vascular graft, balloon, or stent prior to administration to a blood vessel of the subject. The composition may coated on or incorporated into the vascular device using any known suitable method. The composition may be encapsulated in the form of microspheres, nanospheres, microparticles and/or microcapsules, and seeded on or into the vascular device.

In a preferred embodiment for local delivery, the compositions described herein are released from a drug-eluting stent. A typical drug-eluting stent is a peripheral or coronary stent (a scaffold) placed into narrowed, diseased peripheral or coronary arteries that slowly releases a drug. The stent can contain a RAF1 S259A, a nucleic acid encoding RAF1 S259A, an AKT1 inhibitory molecule or an ME-LA construct or a combination thereof instead of. Drug-eluting stents have three parts: a stent platform, a coating, and a drug. The stent itself is generally an expandable metal alloy framework. Many drug-eluting stents are based on a bare-metal stent (BMS). The stents have elaborate mesh-like designs to allow expansion, flexibility and in some cases the ability to make/enlarge side openings for side vessels. A coating, typically of a polymer, holds and elutes (releases) the drug into the arterial wall by contact transfer. Coatings are typically spray coated or dip coated. There can be one to three or more layers in the coating e.g. a base layer for adhesion, a main layer for holding the drug, and sometimes a top coat to slow down the release of the drug and extend its effect.

In other embodiments, the composition is administered systemically. Therapeutic silencing of endogenous genes by systemic administration of siRNAs has been described in the literature (Kim B. et al., American Journal of Pathology, 2004, 165:2177-2185; Soutschek J. et al., Nature, 2004, 432:173-178; Pardridge W. M., Expert Opin. Biol. Ther., 2004, July, 4(7):1103-1113), each of which is incorporated herein by reference in its entirety. In vivo nucleic acid therapy can be accomplished by direct transfer of a functionally active DNA into mammalian somatic tissue or organ in vivo. For example, nucleic acids encoding polypeptides disclosed herein can be administered directly to the tissue of choice. Nucleic acids may also be administered in vivo by viral means. Nucleic acid molecules encoding RAF1S259A proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA, or viral vectors, engineered bacteria may be used as vectors. Nucleic acids may also be delivered by other carriers, including liposomes, polymeric micro- and nanoparticles and polycations such as asialoglycoprotein/polylysine. In addition to virus- and carrier-mediated gene transfer in vivo, physical means well-known in the art can be used for direct transfer of DNA, including administration of plasmid DNA and particle-bombardment mediated gene transfer.

A formulation may be administered in a number of ways depending upon whether local or systemic treatment is desired and upon the area to be treated. The polynucleotide can be administered to the subject either as an oligonucleotide in conjunction with a delivery reagent, or as a recombinant plasmid or viral vector that expresses the polynucleotide. Nucleic acid molecules can be administered to cells by a variety of methods known to those of skill in the art, including, but not restricted to, encapsulation in liposomes, by ionophoresis, or by incorporation into other vehicles, such as hydrogels, biodegradable nanocapsules, and bioadhesive microspheres, or by proteinaceous vectors.

A. Systemic or Local Administration

The formulation can be administered to the subject by any means suitable for delivering the agent to the cells of the tissue at or near the diseased area. For example, a disclosed polynucleotide formulation can be delivered directly to the liver, or can be conjugated to a molecule that targets the liver.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant comprising a porous, non-porous, or gelatinous material).

The formulation can be provided in sustained release composition. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

The polynucleotide formulation can be administered in a single dose or in multiple doses. Certain factors may influence the dosage required to effectively treat a subject, including but not limited to the severity of the disease or disorder, previous treatments, the general health and/or age of the subject, and other diseases present. It will also be appreciated that the effective dosage of the oligonucleotide used for treatment may increase or decrease over the course of a particular treatment. Changes in dosage may result and become apparent from the results of diagnostic assays. For example, the subject can be monitored after administering an oligonucleotide composition. Based on information from the monitoring, an additional amount of the oligonucleotide composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual polynucleotides, and can generally be estimated based on EC50s found to be effective in in vitro and in vivo animal models.

Dosage levels on the order of about 1 mg/kg to 100 mg/kg of body weight per administration are useful in the treatment of a disease. One skilled in the art can also readily determine an appropriate dosage regimen for administering the disclosed polynucleotides to a given subject. For example, the polynucleotides can be administered to the subject once, e.g., as a single injection. Alternatively, the polynucleotides can be administered once or twice daily to a subject for a period of from about three to about twenty-eight days, or from about seven to about ten days.

Thus, the disclosed polynucleotides formulations can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of polynucleotides per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of polynucleotides per kg of bodyweight.

Delivery of a polynucleotide formulation directly to an organ (e.g., directly to the liver) can be at a dosage on the order of about 0.00001 mg to about 3 mg per organ, or preferably about 0.0001-0.001 mg per organ, about 0.03-3.0 mg per organ, about 0.1-3.0 mg per organ or about 0.3-3.0 mg per organ.

Where a dosage regimen involves multiple administrations, it is understood that the effective amount of polynucleotides administered to the subject can include the total amount of polynucleotides administered over the entire dosage regimen. One skilled in the art will appreciate that the exact individual dosages may be adjusted somewhat depending on a variety of factors, including the specific polynucleotides being administered, the time of administration, the route of administration, the nature of the formulation, the rate of excretion, the particular disorder being treated, the severity of the disorder, the pharmacodynamics of the polynucleotides, and the age, sex, weight, and general health of the patient. Wide variations in the necessary dosage level are to be expected in view of the differing efficiencies of the various routes of administration. Variations in these dosage levels can be adjusted using standard empirical routines of optimization, which are well-known in the art. The precise therapeutically effective dosage levels and patterns are preferably determined by the attending physician in consideration of the above-identified factors.

In some embodiments, a subject is administered an initial dose, and one or more maintenance doses of an oligonucleotide formulation. The maintenance dose or doses are generally lower than the initial dose, e.g., one-half less of the initial dose. A maintenance regimen can include treating the subject with a dose or doses ranging from 0.01 mg to 75 mg/kg of body weight per day, e.g., 70, 60, 50, 40, 30, 20, 10, 5, 2, 1, 0.5, 0.1, 0.05, 0.01, 0.005, 0.001, or 0.0005 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

In addition to treating pre-existing diseases or disorders, the oligonucleotide formulations can be administered prophylactically in order to prevent or slow the onset of a particular disease or disorder.

B. Delivery to Cells or Isolated Tissue

In still other embodiments, the composition for increasing RAF1 S259A bioavailability comprises cells expressing a nucleic acid encoding RAF1 S259A, fragment or variant thereof as described above. The nucleic acid molecules may be stably integrated into the genome of the cell or may be maintained in a form extrachromosomally. One approach includes nucleic acid transfer into primary cells in culture followed by autologous transplantation of the ex vivo transformed cells into the host, either systemically or into a particular organ or tissue. Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject.

Standard methods for transfecting cells with nucleic acid molecules are well known to those skilled in the art of molecular biology, see, e.g., WO 94/29469.

The prevent invention will be understood by the following non-limiting examples.

### EXAMPLES

### Materials and Methods

Antibodies and Reagents

Antibodies against p-ERK1/2, ERK1/2, RAFT, pRAF1 S259, pAKT S473, AKT1, AKT2, pan AKT, 14-3-3ζ; Cleaved Notch1, Notch1, DLL4 and Jagged-1 were from Cell Signaling; β-tubulin was home-made; Hemagglutinin (HA) was obtained from Covance; pS6K T389, S6K were obtained from Epitomics; VE-Cadherin, CD31 was obtained from Santa Cruz Biotechnology, Inc. U0126 was obtained from Cell Signaling. LY294002 (a PI3 kinase inhibitor) was obtained from Sigma. VEGF and fibronectin were from R&D.

Cell Culture

Human umbilical vein endothelial cells (HUVEC) and Human umbilical artery endothelial cells (HUAEC) were cultured in medium (Lonza) on fibronectin-coated dishes. Cells at passage 2 to 8 were used for experiment. Bovine aortic endothelial cells (BAECs) were cultured in Dulbecco's Modified Eagles Medium (DMEM) (Lonza) containing 20% fetal bovine serum (FBS) and 100 μg/ml endothelial cell growth supplement (ECGS) (R&D). HEK 293 cells (Human embryonic kidney cells) were cultured in DMEM with 10% FBS. All cells were cultured in 5% CO₂ at 37° C.

Plasmid Construction and Virus Production

HA-tagged human Raf1 (WT and S259A) were amplified from plasmids pMT2-myc-Raf1WT and pMT2-myc-Raf1 S259A (Balan et al., Mol Biol Cell, 17:1141-1153 (2006); Shen et al., Mol Biol Cell, 14:4721-4733 (2003)) (a gift from Guri Tzivion, Wayne State University) using PCR, and were inserted into the NotI site of both pLVX-IRES-puro lentiviral expression vector (Clontech) and pAdTrack shuttle vector (Frank Giordano, Yale). Lentivirus packaging vectors pMDLg/pRRE, pRSV-Rev and pMD2.G were purchased from Addgene Inc. Lentivirus was produced as described in (Dull et al., J. Virol., 72:8463-8471 (1998). To generate adenovirus, HA-Raf1 WT and S259A were cloned from pAdTrack to pAdEasy by homologous recombination in bacteria, and adenoviruses were produced as described before (He et al., Proc. Nat.l Acad. Sci. USA, 95:2509-2514 (1998).

Constitutive ERK2, ERK2-MEK1 (ME) and ERK2-MEK1-LA (LA) plasmids (Robinson et al., Curr Biol, 8:1141-1150 (1998)) were generous gifts from Melanie H. Cobb (University of Texas Southwestern Medical Center). HA-tagged ME and LA were cloned to pAd/CMV/V5-DEST Gateway® adenoviral expression Vector using a Gateway system (Invitrogen). Adenoviruses were generated as manufacturer's instructions.

Lentiviral Infection

Cells were cultured to 50-60% confluence on 6-well plates. Lentivirus was then added to the cells with 5 ug/ml polybrene and centrifuged at 2300 rpm for 90 min at room temperature following a 6-8-hour incubation in 5% CO₂, 37° C. for 6-8 hours. Forty eight hours post infection, cells were harvested for experiment. To obtain stable infected cells, the cells were selected with 0.5 μg/ml puromycin for four days and maintained with 0.1 μg/ml puromycin.

Adenoviral Infection

Adenoviral infection was performed by addition of adenovirus at indicated multiplicity of infection (MOI) to cells cultured at 50-60% for overnight. Cells were used for experiments 48 hours post infection.

### Example 1

### Effect of Blocking RAF1-AKT Crosstalk on ERK Activation in Endothelial Cells

Materials and Methods

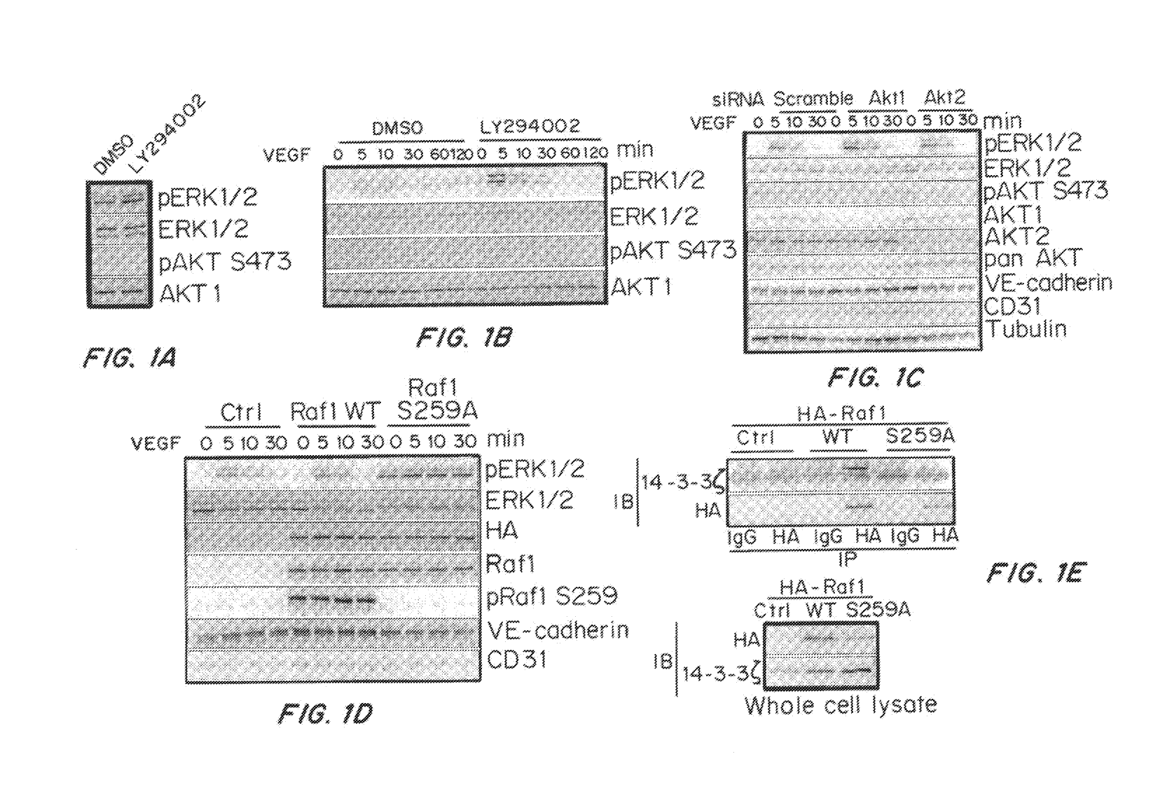

The role of RAF-AKT crosstalk in endothelial cells was investigated using Bovine aortic endothelial (BAEC) cells. BAEC were treated with 10 μM LY294002 (a specific PI3K inhibitor) or DMSO for 30 minutes, under normal culture conditions. Activation of ERK and AKT was analyzed by western blot analysis with indicated antibodies (FIG. 1A).

The effect of PI3K inhibition on ERK activation by an angiogenic factor such as VEGF was also investigated. BAEC cells serum-starved for overnight, were pretreated either with DMSO or 10 μM LY294002 for 30 minutes. The cells were then stimulated with 50 ng/ml VEGF for indicated times and activation of ERK and AKT was analyzed by western blot with indicated antibodies (FIG. 1B).

For Western blot analysis, cells were washed three times with ice cold PBS and lysed in RIPA buffer supplemented with protease (Sigma) and phosphatase (Boston Bioproducts) inhibitors. The cells lysates were then subjected to SDS-PAGE and Western Blot.

To determine if the RAF1-AKT cross talk was AKT isoform specific, HUAEC cells were transfected with 5 nmol control scramble, Akt1 and Akt2 siRNAs.

Human Akt1, Akt2 Flexitube siRNAs and AllStars Negative Control siRNA were purchase from Qiagen, Inc. Cells cultured in 6-well plates were transfected at 50% confluence with 5 nmol siRNA, using TransPass R2 Transfection Reagent (NEB) following the manufacturer's instructions. Forty eight hours post siRNA transfection, the cells were serum starved for overnight and stimulated with 50 ng/ml VEGF for indicated times and analyzed by western blot with indicated antibodies (FIG. 1C). Knocking down efficiency was determined by qPCR or Western blot analysis.

To determine whether Ser259 to Ala259 mutation in RAF1 affects ERK activation in endothelial cells, wild type and mutant RAF1 (RAF1 S259A) were overexpressed in HUVEC. HUVEC cells were infected with equal amount of empty lentivirus (Control), RAF1 WT (Raf1 WT) lentivirus or RAF1 S259A (Raf1 S259A) lentivirus as described above. Forty eight hours later, the cells were selected with 0.5 μg/ml puromycin for 4 days and analyzed by western blot with indicated antibodies. HUVEC cells described above were lysed as described, and immunoprecipitation was performed using anti-HA or control mouse IgG. For immunoprecipitation, cells were otherwise lysed in Triton lysis buffer (10 mM Tris-HCl, pH 7.5, 150 mM NaCl, 1% Triton X-100) supplemented with protease (Sigma) and phosphatase (Boston Bioproducts) inhibitors. Antibodies and Protein G agarose beads were then added to the cell lysates and incubated for 4 hours and 2 hours respectively. The beads were washed 6 times with Triton lysis buffer, boiled in 1× Laemmli loading dye, and subjected to SDS-PAGE and Western Blot

The immunoprecipitates were then analyzed by western blot with indicated antibodies.

Results

As shown in FIGS. 1A-1B, Western Blots showing ERK and AKT activation in BAEC cells treated with 10 μM LY294002 or DMSO shows activation of ERK and AKT in VEGF-stimulated BAEC pretreated either with DMSO or 10 μM LY294002. Western Blot showed ERK and AKT activation in VEGF-stimulated HUAEC cells transfected control scramble, Akt1 and Akt2 siRNAs (FIG. 1C). As shown by FIGS. 1D-1E, Western blot showed ERK activation in HUVEC cells were infected with equal amount of empty control, RAF1 WT (Raf1 WT) or RAF1 S259A (Raf1 S259A) lentivirus. Western Blot showed immunoprecipitation of 14-3-3 in HUVEC cells infected with equal amount of empty control, RAF1 WT (Raf1 WT) or RAF1 S259A (Raf1 S259A) lentivirus.

A modest increase in ERK activity was observed following LY294002 treatment. A significant increase in VEGF-induced ERK activation was observed in LY294002 treated cells.

Since PI3K-AKT pathway crosstalks with Raf1-MEK-ERK pathway at the AKT-RAF1 level, both Akt1 and Akt2 in HUAEC cells were knocked down using siRNA, in order to determine which AKT isoform is involved in the crosstalk in endothelial cells. Knocking down Akt1 modestly increased ERK activity, but knocking down Akt2 did not.

Application of PI3K inhibitors such as LY294002 inhibits activation of all the isoforms of AKT and presents with specificity concerns. AKT has been shown to phosphorylate RAF1 at Ser259, which in turn recruits 14-3-3. These chain of events inhibit RAF 1-mediated activation of ERK (Zimmermann and Moelling, Science, 286:1741-1744 (1999)). Thus a RAF1 S259A mutant which mimics non-phosphorylated RAF1 at the Ser259 site was used to block the crosstalk between RAF1 and AKT. Consistent with previous findings, wild type RAF1 co-immunoprecipitated with 14-3-3 while RAF1 S259A mutant did not. Thus, mutation of Ser259 specifically blocked RAF1-AKT crosstalk.

To determine whether Ser259 to Ala259 mutation in RAF1 affects ERK activation in endothelial cells, wild type and mutant RAF1 (RAF1 S259A) were overexpressed in HUVEC. The data shows that overexpression of RAF1S259A caused constitutive activation of ERK. Overexpression of RAF1 WT only had a marginal effect on ERK activation. These results demonstrate that the effective of RAF1 S259A on ERK activation was specifically caused by blocking of RAF1-AKT crosstalk rather than protein overexpression per se.

In summary, blocking RAF1-AKT crosstalk by mutating RAF1 Ser 259 to Ala259 results in constitutive ERK activation in endothelial cells.

### Example 2

### Effect of Blocking RAF1-AKT Crosstalk on Tube Formation, Cell Migration, Survival and Cell Proliferation

ERK activation has been shown to play important roles in a variety of cellular events such as proliferation, migration, apoptosis, etc. Given the constitutive ERK activation in RAF1 S259A overexpressed endothelial cells, the effect of blocking RAF1-AKT crosstalk on endothelial tube formation, cell proliferation, survival and migration was investigated.

The ability of endothelial cells to form tubes in vitro was investigated using a Matrigel tube formation assay, a process which mimics blood vessel formation in vivo which is essential for angiogenesis and arteriogenesis as well.

Matrigel Tube Formation Assay

Twenty four well plates were coated with 0.5 ml growth factor reduced Matrigel (BD Bioscience) at 37° C. for 1 hour. Endothelial cells were then trypsinized with 0.25% Trypsin-EDTA (Gibco), washed twice with and resuspended in serum-free DMEM (Lonza). Cell number was counted, and 6×10⁴ cells were plated onto the Matrigel coated plates. Endothelial tube was allowed to form for 8 hours. Then the cells were fixed in 4% paraformaldehyde (PFA) in PBS, and pictures were taken under a convert microscope. Tube length was quantitated using Image J 1.410 (NIH).

Migration Assay

For the cell migration assay, endothelial cells were cultured to confluence in 24-well plates. Prior to serum starvation, the cells were treated with 10 μg/ml mitomycin C for four hours to block proliferation, and then washed three times with DMEM. The cells were starved overnight with 0.5% FBS, DMEM. A scratch was then made using a 200 μl tip, and the cells were allowed to migrate for 18 hours in DMEM containing 0.5%, 10% FBS or 50 ng/ml VEGF. Pictures were taken right after scratch making and at the end of migration. The migration rate was then determined by analyzing the distance that cells migrated during the period.

Proliferation Assay

Endothelial cells were placed on fibronectin-coated 24-well plates at a seeding density of 6000 cells/well in EGM-2 medium. Cell proliferation was analyzed by counting cell numbers every 24 fours.

Results

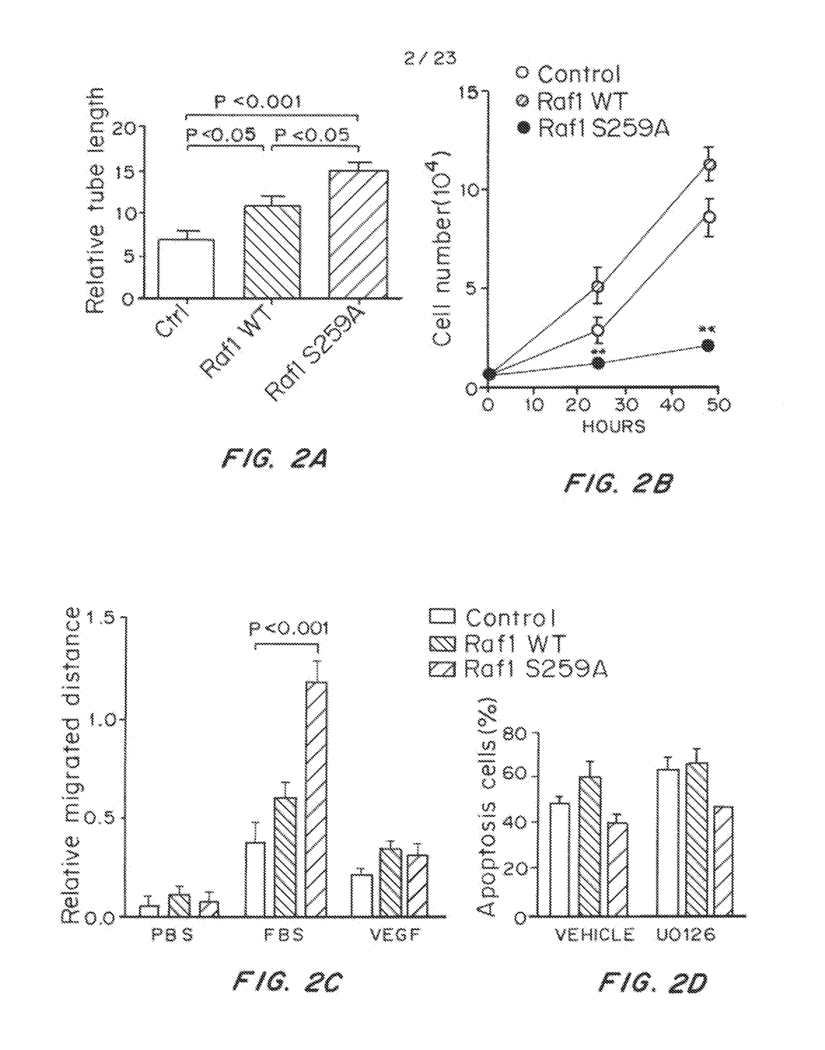

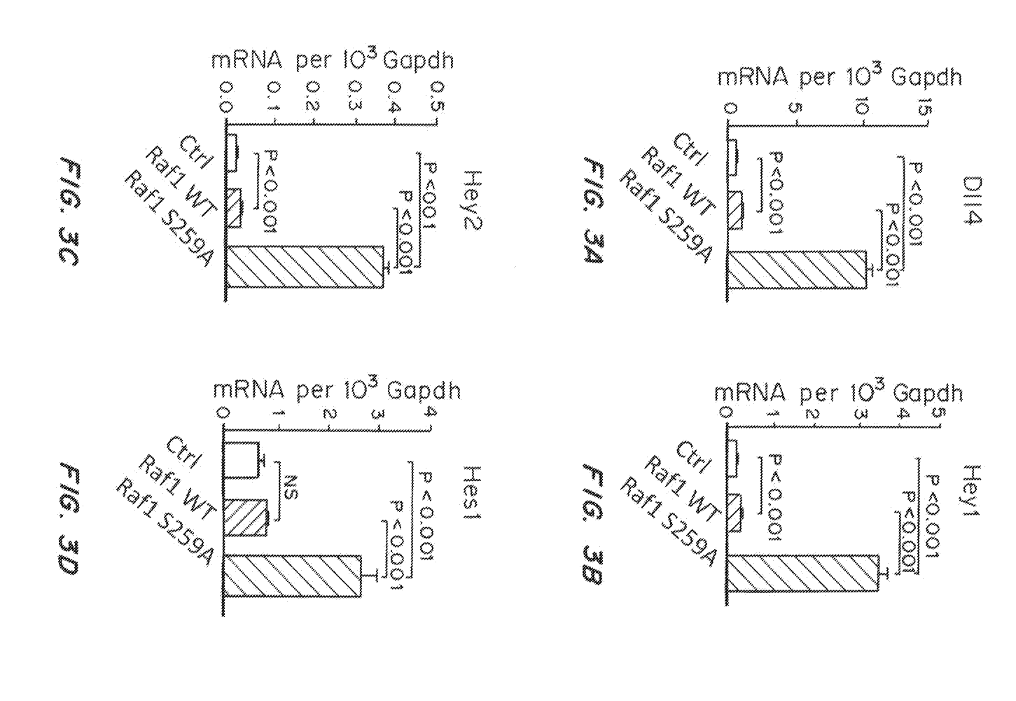

Overexpression of RAF1 S259A in HUVEC significantly improved tube formation (FIG. 2A). Wild type RAF1 also slightly improved tube formation. This is likely due to the modest increase in ERK activity. Unexpectedly, while overexpression of RAF 1 WT had little effect on HUVEC proliferation, RAF 1 S259A significantly inhibited cell proliferation (FIG. 2B).

Cell migration was analyzed using a wound healing assay. As shown in FIG. 2C, RAF1 S259A significantly promoted cell migration under stimulation of serum, while RAF1 WT had no obvious effect.

Both RAF 1 and ERK have been indicated to play important roles in endothelial cell survival (Alavi et al., Science, 301:94-96, (2003); Sridhar et al., Molecular Cancer Therapeutics, 4:677-685 (2005)). Consequently, the effect of overexpression of RAF1 S259A on cell survival was investigated. To induce apoptosis, cells were serum-starved for 48 hours and apoptotic cells were visualized by flow cytometry as described below.

Apoptosis Assay

Endothelial cells were cultured in fibronectin-coated 6-well plates until 70-80% confluent. Apoptosis was induced by serum withdrawal for 48 hours. Where indicated, cells were treated with 10 μM U0126 (1,4-dimino-2,3-dicyano-1,4-bis[2-aminophenylthio]butadiene), a MEK1 and MEK 2 selective inhibitor, dissolved in dimethyl sulfoxide or an equal volume of dimethyl sulfoxide (DMSO) as control for U0126 treatment. Both floated and attached cells were harvested. Then apoptotic cells were stained with Annexin V-Allophycocyanin (APC) and propidium iodide (PI) using an Apoptosis analysis kit (eBioscience), and analyzed by flow cytometry Annexin V positive and PI negative cells were defined as apoptotic cells.

The data shows that cells overexpressing RAF1 S259A showed significantly higher survival compared to cells infected with an empty control lentivirus, while RAF1 WT overexpressed cells showed lower survival (FIG. 2D, “Vehicle”) as measured by the percent of apoptotic cells.

In addition, partial inhibition of ERK using an MEK inhibitor, U0126 significantly inhibited the protection of cell survival by RAF1 S259A (FIG. 2D, “U0126”) suggesting that this effect is at least partially ERK-dependent. In summary, the data shows that blocking of RAF1-AKT crosstalk promotes tube formation, cell migration and survival, and inhibits proliferation.

### Example 3

### Effect of Blocking RAF1-AKT Crosstalk on Dll4-Notch Pathway Activation and Arterial Endothelial Gene Expression

Arterialization of a subset of capillary vasculature is required for arteriogenesis in adult tissues (Carmeliet, Nat Med, 6:389-395 (2000); Simons, Methods Enzymol, 445:331-342 (2008)). Also, arterialization is an essential process in artery-vein specification during blood vessel development (Carmeliet, Nat Med, 6:389-395 (2000)). RAF1-AKT crosstalk has been shown to govern artery-vein specification in zebrafish, where a higher RAF1-MEK-ERK activity favors endothelial artery fate (Hong et al., Circ Res, 103:573-579 (2008); Hong et al., Curr Biol., 16:1366-1372 (2006)). However, in vitro studies in mammalian endothelial cells have revealed that VEGF or FoxC1/2-induced arterial endothelial cell marker expression is independent of ERK activity, showing that rather, it is dependent on PI3K-AKT pathway. On the contrary, in vivo studies have revealed aortic-specific ERK activation in mouse embryos, indicating a role of the RAF1-MEK-ERK consensus in artery development (Corson et al., Development, 130:4527-4537 (2003)). Blocking the RAF1-AKT crosstalk by mutating a single amino acid of RAF1 as described herein was used to determine the specific role of RAF1-AKT crosstalk in arterial marker expression and eventually in aterialization.

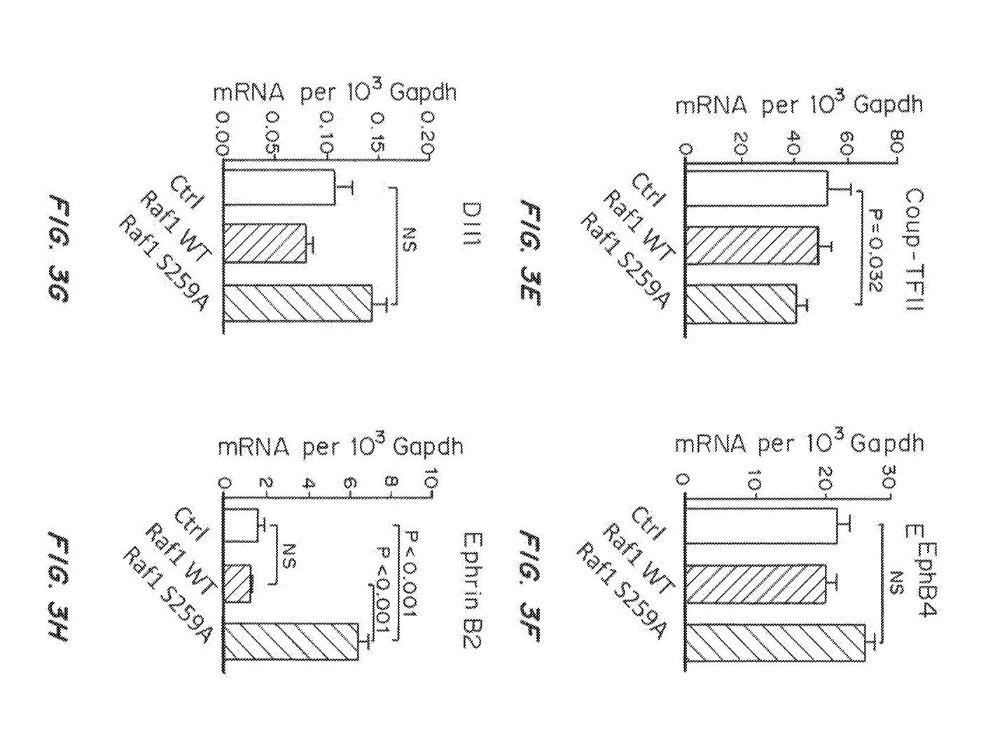

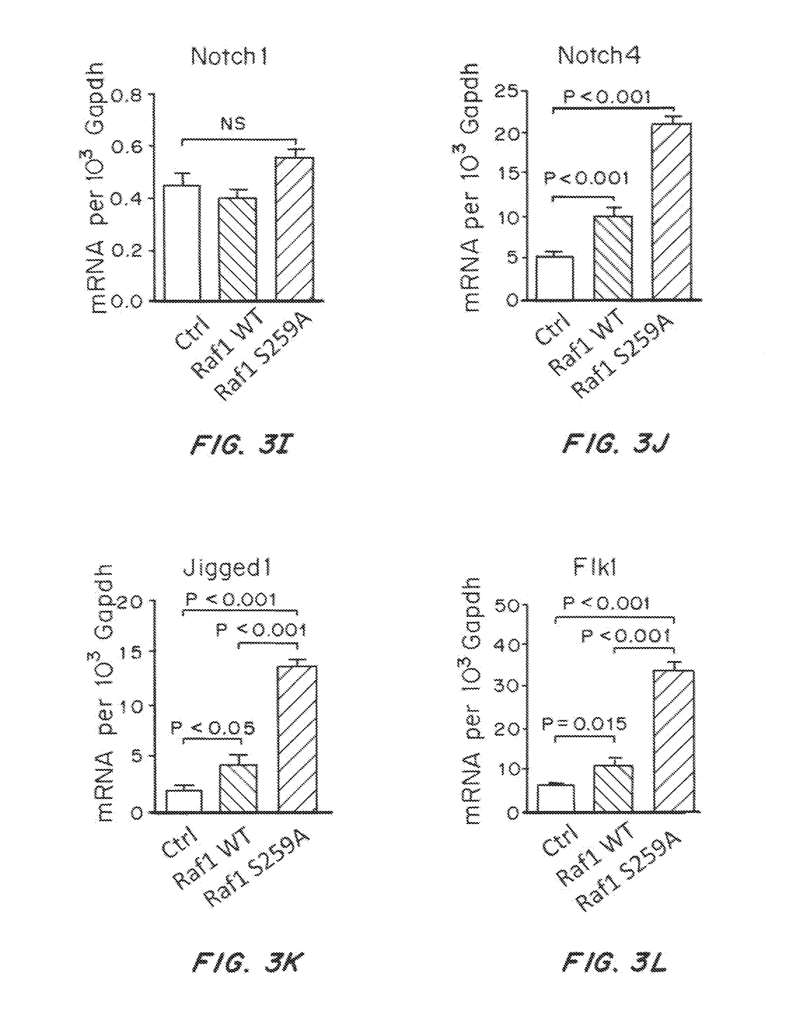

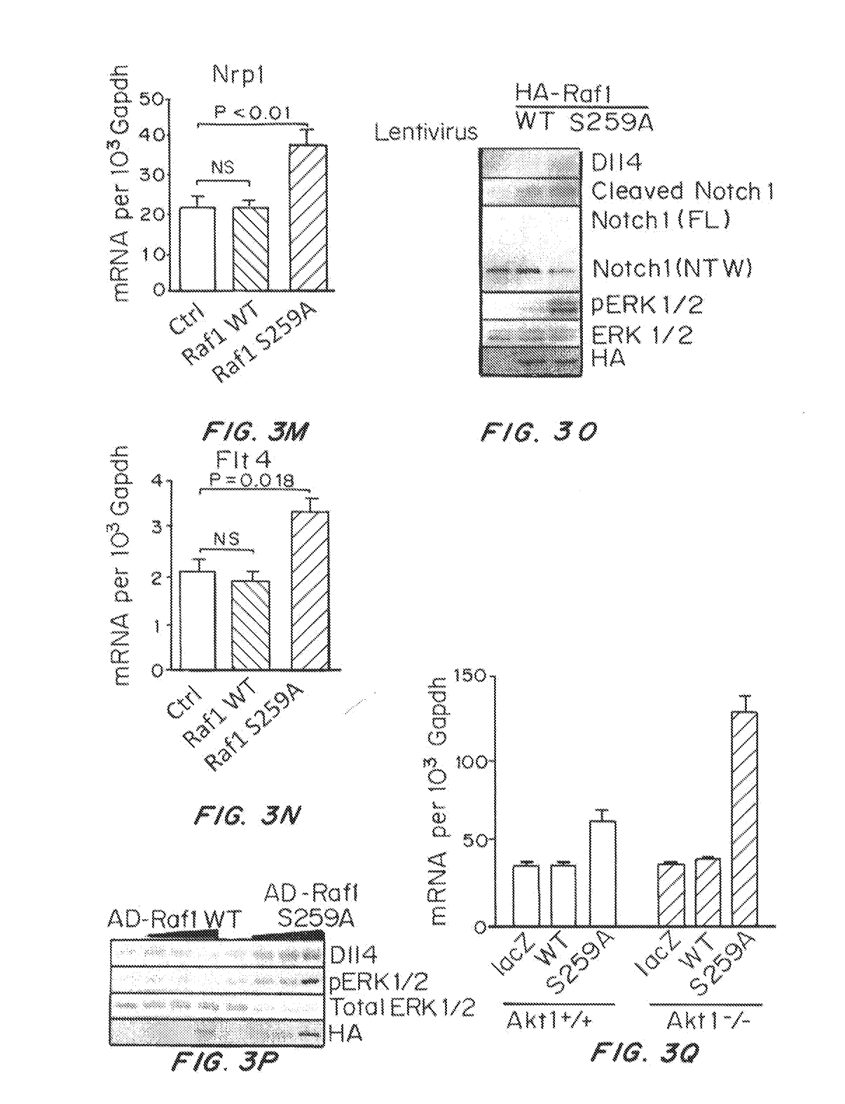

The expression levels of a collection of genes involved in artery-vein specification in control, Raf1 WT and Raf1 S259A lentivirus infected HUVEC were investigated using real time quantitative PCR. For qPCR analysis, RNA was prepared from cells using a Qiagen RNaeasy kit (Cat. 74106) following the instructions from the manufacturer. cDNA was synthesized using iScript cDNA Synthesis kit (Bio-Rad). Gene expression was then analyzed by qPCR using a Bio-Rad SYBR kit on a Bio-Rad CFX96 system. Primers for qPCR were all purchased from SA Biosciences Corporation, MD. These are listed in Table 1. All gene expressions were normalized to glyceraldehyde-3-phosphate dehydrogenase (GAPDH) levels and represented as mRNA per 10³ GAPDH. Standard errors were calculated based on three independent experiments. The cell lysates of these cells were also prepared and analyzed by western blot using indicated antibodies. In other experiments, HUVEC cells were infected with different doses (MOI: 0, 5, 10, 50) of Raf1 WT or S259A adenovirus for 48 hours. Cells were lysed and analyzed by western blot using indicated antibodies. (FIG. 3P).

TABLE 1
List of primers used for qPCR.

| | |
|---|---|
| hSox18 fwr | CTTCATGGTGTGGGCAAAG (SEQ ID NO: 1) |
| hSox18 rev | GCGTTCAGCTCCTTCCAC (SEQ ID NO: 2) |
| hSox7 fwr | CAGCAAGATGCTGGGAAAGT (SEQ ID NO: 3) |
| hSox7 rev | GTTGGGGTAGTCCTGCATGT (SEQ ID NO: 4) |
| hGapdh fwr | TGCACCACCAACTGCTTAGC (SEQ ID NO: 5) |
| hGapdh rev | GGCATGGACTGTGGTCATGAG (SEQ ID NO: 6) |
| hCoup-TFII | GCAAGTGGAGAAGCTCAAGG (SEQ ID NO: 7) |
| fwr | |
| hCoup-TFII | GCTTTCCACATGGGCTACAT (SEQ ID NO: 8) |
| rev | |
| hLyve1 fwr | ACTTCCATCTGGACCACGAG (SEQ ID NO: 9) |
| hLyve1 rev | AGCCTACAGGCCTCCTTAGC (SEQ ID NO: 10) |
| hSox17 fwr | CAGAATCCAGACCTGCACAA (SEQ ID NO: 11) |
| hSox17 rev | GCGGCCGGTACTTGTAGTT (SEQ ID NO: 12) |
| hProx1 fwr | GGCATTGAAAAACTCCCGTA (SEQ ID NO: 13) |
| hProx1 rev | ACAGGGCTCTGAACATGCAC (SEQ ID NO: 14) |
| hVegfr3 fwr | GGTGTCGATGACGTGTGACT (SEQ ID NO: 15) |
| hVegfr3 rev | CTCTGCCTGGGACTCCTG (SEQ ID NO: 16) |
| hRaf1 fwr | ACCCATTCAGTTTCCAGTCG (SEQ ID NO: 17) |
| hRaf1 rev | GCTACCAGCCTCTTCATTGC (SEQ ID NO: 18) |
| mRaf1 fwr | CAAGTGGCATGGAGATGTTG (SEQ ID NO: 19) |
| mRaf1 rev | CGATTGTGACTCAGTGGTGT (SEQ ID NO: 20) |
| mSox18 fwr | AACAAAATCCGGATCTGCAC (SEQ ID NO: 21) |
| mSox18 rev | CGAGGCCGGTACTTGTAGTT (SEQ ID NO: 22) |
| mVegfr3 fwr | GCTGTTGGTTGGAGAGAAGC (SEQ ID NO: 23) |
| mVegfr3 rev | TGCTGGAGAGTTCTGTGTGG (SEQ ID NO: 24) |
| mGapdh fwr | AACTTTGGCATTGTGGAAGG (SEQ ID NO: 25) |
| mGapdh rev | ACACATTGGGGGTAGGAACA (SEQ ID NO: 26) |

Results

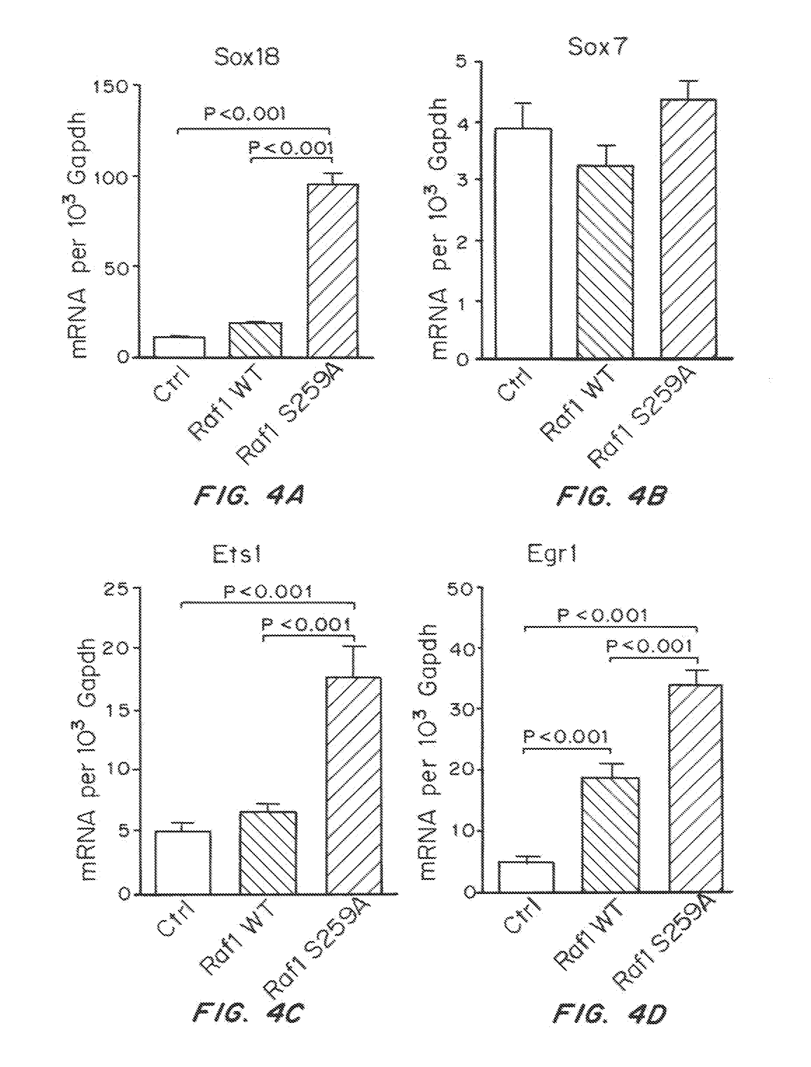

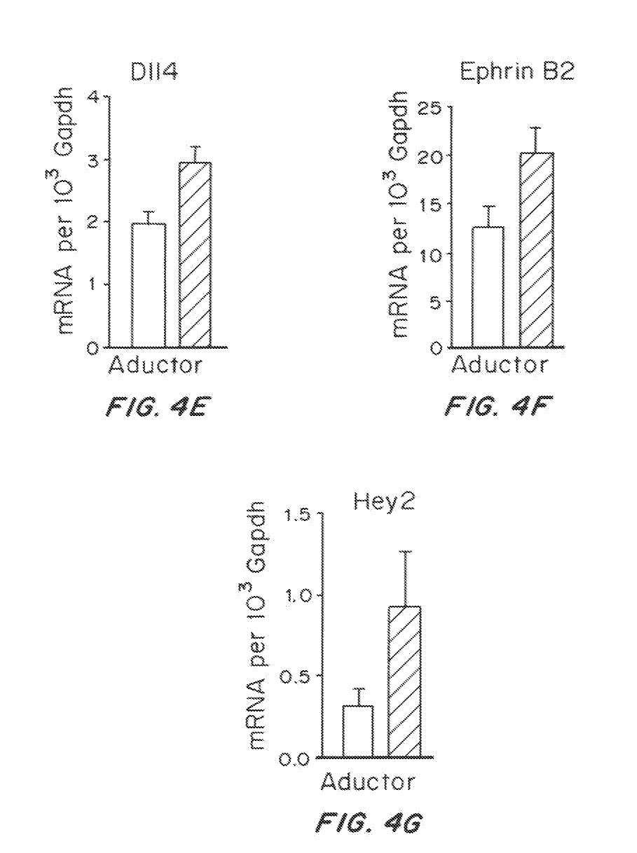

Overexpression of RAF1S259A in HUVEC dramatically induced the arterial endothelial marker genes, Dll4 (FIG. 3A), Hey1 (FIG. 3B), Hey2 (FIG. 3C), Hes1 (FIG. 3D) Ephrin B2 (FIG. 3H), Notch4 (FIG. 3J), Jagged1 (FIG. 3K), and neuropilin1 (FIG. 3M) (Nrp1) while slightly inhibiting the venous marker gene Coup-TFII (FIG. 3E). Similar to Dll4, Flk1 (FIG. 3L) and Flt4 (FIG. 4N], two genes showed to be involved in tip cell formation were also induced by RAF1 S259A. Thus, RAF1 S259A induced expression of all genes thought to be involved in arterial fate determination while there were no changes in expression of venous fate genes COUP-IIF (FIG. 3E), EphB4 (FIG. 3F).

Consistent with the qPCR results, Dll4 protein also dramatically increased in RAF1 S259A overexpressed HUVEC as shown by western blot (FIG. 3O). In contrast, RAF1 WT only had a marginal effect on these genes. Expression of Dll1, a homologue of Dll4 which is also specifically expressed in arteries and involved in arteriogenesis, was not affected (FIG. 3G), suggesting that the effect of RAF1 S259A on Dll4 expression is specific. Furthermore, when transiently infected with different doses of RAF1 expression adenovirus, HUVECs overexpressing RAF1 S259A showed a dose-dependent and parallel increase in both DLL4 protein and ERK activity (FIG. 3P). Again, RAF1 WT only had a marginal effect.

Hey1, Hey2, Hes1 and Ephrin B2 are all known downstream targets of the Dll4-Notch signaling. Hence, RAF1 S259A very likely primarily activates the Dll4-Notch signaling, which in turn turns on these downstream target genes. Consistent with this presumption, NOTCH1 was activated by RAF1 S259A indicated by western blot of cleaved NOTCH1, an active form of NOTCH1 (FIG. 3O). Taken together, these results strongly suggested that block of RAF1-AKT crosstalk induces arterial gene expression by activating the DLL4-NOTCH signaling and possibly promotes arterialization. Furthermore, Raf1 SA strongly induced DLL4 expression in Akt1^{-/-} demonstrating that this is not a Pi3K/Akt-dependent event (FIG. 3Q).

In addition to arterial fate genes, Raf1SA introduction also activated expression of key transcription factors thought to be involved in arterial specification—Sox18, Ets1 and Egr1 while expression of a Sox factor not involved in this process (Sox 7) was not changed (FIGS. 4A-D). Introduction of Ad-RAF1S259A construct into the femoral artery induced qPCR-determined Dll4, Ephrin B2 and Hey 2 expression (FIGS. 4E-G).

### Example 4

### Role of ERK Activity in Arterial Gene Expression

Controversial results have been reported regarding the role of RAF-MEK-ERK and PI3K-AKT pathways in regulation of arterial gene expression. ERK activity has been implicated as essential for arterial gene expression in vivo but not in vitro (Hayashi and Kume, PLoS One, 3:e2401 (2008); Hong et al., Circ Res, 103:573-579 (2008); Liu et al., Mol Cell Biol, 23:14-25 (2003)). One possible explanation is difference between in vitro and in vivo models used to study ERK activation. ERK activation in vivo is measured over the course of several hours, a far longer time frame than the transient ERK activation observed in cultured endothelial cells following VEGF stimulation. Thus, there appear to be important fundamental differences in ERK activation between in vivo and in vitro models. The constitutively active ERK system developed in these studies (by blocking the RAF1-AKT crosstalk) was used to determine whether prolonged ERK activation induces arterial gene expression.

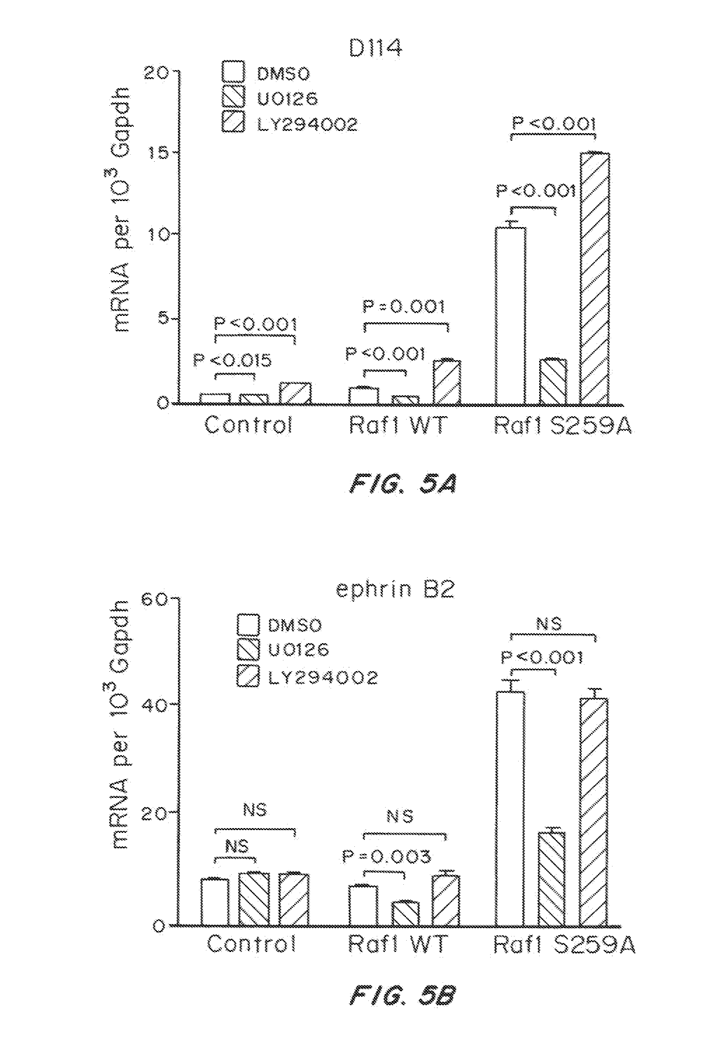

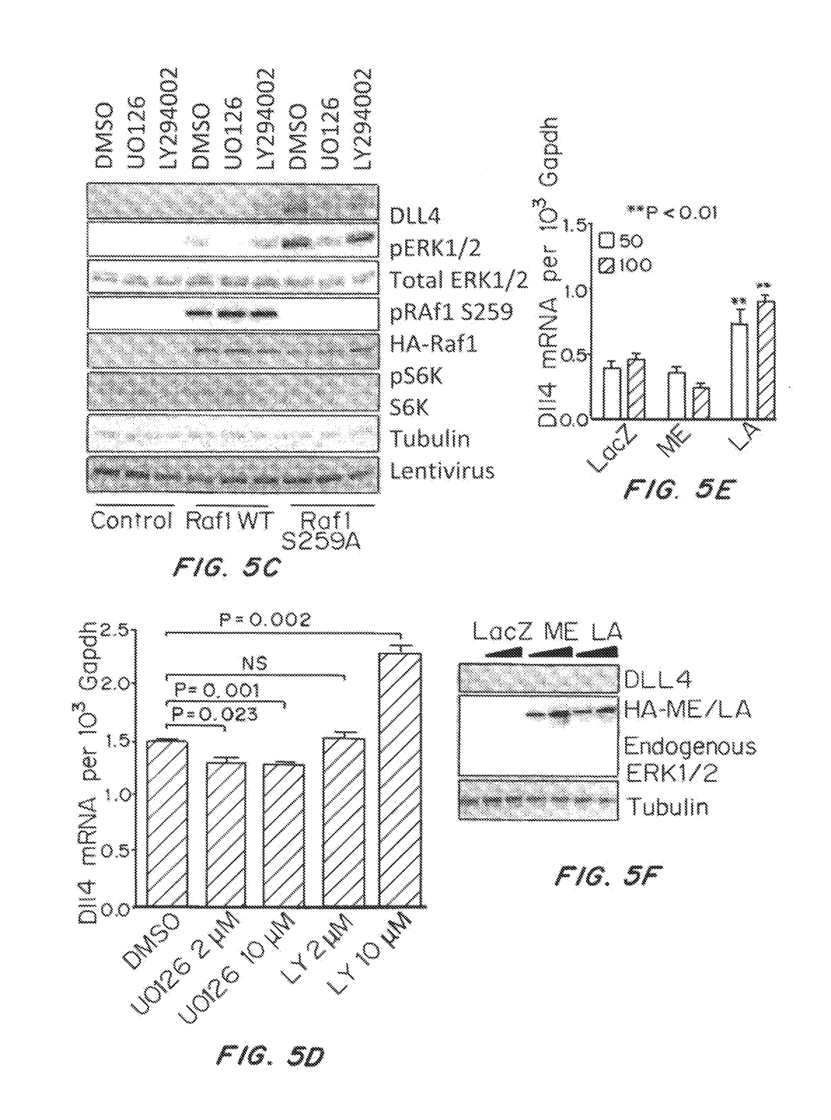

To determine whether ERK activity is required for RAF1-AKT crosstalk blockade-induced arterial gene expression, HUVECs infected with control, RAF1 WT and RAF1 S259A lentivirus were treated with the MEK inhibitor, U0126 to inhibit ERK activation. HUVECs were also treated with LY294002 (a PI3K inhibitor). Specifically, HUVEC infected with control, Raf1 WT, or Raf1 S259A lentivirus were treated with 10 μM/ml U0126, 10 μM LY294002 or equal volume of DMSO for 24 hours. The expression of Dll4 and EphrinB2 was then analyzed by qPCR as described above, and by western blot using indicated antibodies (FIG. 5C).

In other experiments, HUVECs were treated with 2 and 10 μM U0126, 2 and 10 μM LY294002 or equal volume of DMSO for 24 hours and Dll4 gene expression was determined by qPCR. HUVEC cells were infected with lacZ, ME or LA adenovirus at MOI 50 and 100 for 48 hours. Dll4 gene expression was then analyzed by both qPCR (5E) and western blot (5F). Standard errors were calculated based on three independent experiments.

qPCR results showed that inhibition of MEK (but not PI3K) almost completely abolished Dll4 (FIG. 4A) and Ephrin B2 (FIG. 5B) expression induced by RAF1 S259A. In addition, inhibition of ERK also inhibited Dll4 and Ephrin B2 expression in control (vehicle lentivirus) and RAF1 WT cells. This phenomenon was also confirmed at the protein level by Western blot (FIG. 5C). Noticeably, both Dll4 mRNA and protein levels perfectly correlated with the level of ERK activation. For example, application of U0126 did not completely abolish ERK activation in RAF1 S259A cells but partially inhibited it to a level similar to that in untreated RAF1 WT cells. In parallel, there are almost the same levels of Dll4 mRNA and protein in these two groups of cells. These results show that ERK activity is essential for RAF1 A256A-induced Dll4 gene expression.

The data showed that inhibition of PI3K-AKT pathway resulted in increased ERK activity in endothelial cells. To determine whether this ERK activity increase accounts for Dll4 expression, cells were treated with a PI3K inhibitor, LY294002 and measured Dll4 levels by both qPCR and western blot analysis. As shown in FIGS. 4A and C, inhibition of PI3K-AKT pathway caused modest but significant increase in Dll4 mRNA and protein levels in all the cells. Again, this increase correlated with the slight increase of ERK activity. Wild type endothelial cells were treated with different doses of U0126 and LY294002, and a dose-dependent effect on Dll4 gene expression observed as well (FIG. 5D).

To further confirm the essential role of ERK in Dll4 expression, two constitutive ERK constructs: ME and LA were overexpressed in HUVEC by adenovirus infection (see Robinson, et al. Curr. Biol. 9(21):1141-1150 (1998)). The difference between ME and LA is that four lysines in the nuclear export sequence of MEK1 were mutated to alanines in LA construct, which enables nuclear localization of LA while ME stays in cytosol. HUVEC cells were treated with 2 and 10 μM U0126, 10 μM LY294002 or equal volume of DMSO for 24 hours and Dll4 expression was determined in these cells by both qPCR and Western blot. A significant increase in Dll4 expression was observed in LA but not ME overexpressed cells (FIGS. 5E, F). These results indicated that both ERK activation and its nuclear localization are required. The collective data from these studies show that ERK activation, very likely a prolonged ERK activation controlled by RAF1-AKT crosstalk, is essential for arterial gene expression.

In summary, blocking of RAF1-AKT crosstalk either by inhibition of the PI3K-AKT axis or by a single amino acid mutation on the AKT phosphorylation site of RAF1 increased ERK activation, promoted tube formation, cell migration and survival, and inhibited cell proliferation in endothelial cells. Increased ERK activation either by overexpression of RAF1 S259A, inhibition of PI3K-AKT pathway or introduction of constitutive active ERK all induced arterial genes, particularly Dll4 expression. In conclusion, ERK activity determined by Raf1-AKT crosstalk modulates arteriogenesis in vitro by regulating DLL4-Notch signaling.

### Example 5

### Endothelial ERK Signaling Controls Lymphatic Fate Specification in a Noonan Syndrome Mouse Model

One of the more common and least understood lymphatic defects is lymphangiectasia, a pathological dilation of dysmorphic lymphatic vasculature that can lead to lymphedema and compression of nearby structures (Faul, Am J Respir Crit Care Med 161:1037-1046 (2000); Adams, et al. Nat Rev Mol Cell Biol 8:464-478 (2007)). Lymphatic defects such as lymphangiectasia can be particularly prominent in patients with Noonan and LEOPARD syndromes, conditions characterized by gain-of-function mutations in the RAS/RAF signaling cascade (Aoki, et al. Hum Mutat 29:992-1006 (2007); Tidyman, et al. Curr Opin Genet Dev 19:230-236.5, 6 (2009)). The molecular basis of the lymphatic defects in these diseases is still unknown.

Lymphatic vessels are thought to arise from PROX1-positive endothelial cells in the cardinal vein in response to induction of SOX18 expression. However, the molecular event responsible for increased SOX18 expression has not been established. An endothelial-specific non-AKT suppressible Raf1 mutant transgenic mouse model was used to show that the RAF1-MEK-ERK signaling input regulates Sox18-induced LEC fate specification and developmental lymphangiogenesis. Activation of ERK signaling in blood endothelial cells, using a mutant Raf1 gene that cannot be shut down by PI3-kinase signaling (RAF1S259A), induces a persistent induction of SOX18 and PROX1 expression. This in turn leads to development of lymphangiectasia highly reminiscent of abnormal lymphatics seen in Noonan syndrome and similar “Rasopathies”. These data establish that ERK activation plays a key role in lymphatic endothelial cell fate specification and that excessive ERK activation is the basis of lymphatic abnormalities seen in Noonan syndrome and related diseases.

Methods and Materials

### Cell Culture.

Human Umbilical Vein Endothelial Cells (HUVEC), Human Umbilical Artery Endothelial Cells (HUAEC) and HMVEC-dLyAd-Adult Human Dermal Lymphatic Microvascular Endothelial Cells (HDLEC) were purchased from Lonza. HUVEC and HUAEC were cultured in M199 medium supplemented with 20% FBS, 100 μg/ml ECGS and 100 m/ml heparin (Sigma). HDLECs were cultured in EBM-2MV medium (Lonza). 293 cells were maintained in DMEM medium containing 10% FBS.

### Antibodies and Reagents.

The following antibodies were used for Western blot. Anti-pERK1/2, ERK1/2, pRAF1S259, RAFT (Cell Signaling); anti-VE-cadherin, CD31 (Santa Cruz), anti-HA (Covance). Antibodies used for immunofluorescence staining are anti-CD31 Rabbit polyclonal, anti-β-GAL (Abcam), anti-CD31 Rat (BD Biosciences), anti-VEGFR3, anti-neuropilin 1, anti-COUP-TFII (R&D), anti-SMA (Sigma), anti-connexin 40 (Alpha Diagnostic), anti-SOX18 (Aviva), anti-PROX1 (Angiobio), anti-Ki67 (Dako), anti-LYVE1 (Abcam). Secondary antibodies for immunoblotting were from Sigma, for immunohistochemistry from Invitrogen. Blocking Reagent for immunofluorescence staining was from Perkin Elmer. U0126 and Rapamycin were from Cell Signaling. LY294002 was from Sigma. AKT inhibitor VIII was from Calbiochem. VEGF-A164 and VEGF-C were from R&D.

### Transgenic Mice.

Human wild type Raf1 and RAF1S259A plasmids were gifts of Dr. Guri Tzivion (Wayne State University). To generate RAF1S259A transgenic mice, hRAF1S259A was cloned to NotI+PstI sites of pBI-G Tet-off vector (Clontech). TRE-RAF1S259A transgenic mouse line was then generated at the Yale Animal Genomics Services center. VE-cadherin-tT A mice (Sun, et al. Proc Natl Acad Sci USA 102:128-133 (2005)) were from W. Sessa lab (Yale). Time-mating was setup by intercross of male TRE-RAF1 S259A mice with VE-cadherin-tTA female mice. For paraffin and frozen sections, embryos were fixed in 4% paraformaldehyde for overnight at 4° C.

### X-Gal Staining.

X-gal staining was performed using a β-Gal Expression In Tissue kit from Millipore following the manufacturer's instructions.

### Immunohistochemistry.

H&E staining was performed as described by Kiernan, J. A. Histological and histochemical methods: theory and practice. UK: Scion: Bloxham (2008). Immunofluorescence staining of paraffin or frozen sections was performed by incubation of primary and secondary antibodies in 0.5% Blocking Reagents in TNT buffer (50 mM Tris-HCl pH 7.5, 150 mM NaCl, 0.05% TWEEN®-20) Fluorescent images were taken using Volocity 6.0 on a Nikon eclipse Ti Spinning Disk Microscope (Perkin Elmer). H&E images were taken with a Nikon eclipse 80i Microscope.

### Whole Mount Immunostaining.

Embryonic tissues were fixed in 4% PFA overnight at 4° C. followed by 3 washings with PBS. The tissues were then permeabilized and blocked in TNBT buffer (100 mM Tris-HCl, pH7.5, 150 mM NaCl, 0.5% Triton-X100 and 0.05% Blocking Reagent) overnight at 4° C. and washed with TNT (100 mM Tris-HCl, pH7.5, 150 mM NaCl, 0.5% Triton-X100) buffer for 6 times at room temperature. Next, the tissues were stained with primary antibodies at 4° C. followed by fluorescent-labeled secondary antibodies in TNBT for 2 hours at room temperature. After six washings with TNT buffer, the tissues were mounted with Gold Prolong Mounting reagent (Invitrogen).

### Image Analysis.

Diameter of vessels was analyzed with Biologic CMM Analyzer as described by Ruifrok, et al Anal Quant Cytol Histol 23:291-299 (2001). Lumen area of jugular veins was analyzed using Image J.

### Plasmids.

HA-Raf1 WT and S259A were amplified by PCR from pMT2-myc-Raf1 WT and S259A. They were the inserted into NotI site of the pLVX-IRES-Puro (Clontech) and pAd-Track-CMV to generate pLVX-HA-Raf1 WT, S259A and pAD-HA-Raf1 WT, S259A constructs. HA-Raf1 WT and S259A were cloned to pAdEasy-1 by recombination of pAD-HA-Raf1 WT, S259A and pAdEasy-1 as described by Ruifrok 2001; Luo, et al. Nat Protoc 2:1236-1247 (2007). HA-ME and LA were amplified by PCR from pEntry-ME and LA respectively. They were then constructed to pENTER/D-TOPO (Invitrogen). HA-ME and LA were then cloned to adenovirus vector pAD/CMV/V5-DEST (Invitrogen) by recombination as instructed by the company's manual.

### Production and Infection of Lentiviruses and Adenoviruses.

Raf1 lentiviruses were produced in 293A cells (Invitrogen) by co-transfection of pLVX-HA-Raf1 WT or S259A with lentiviral packaging plasmids pMD2.G, pRSV-REV and pMDLg/pRRE (Addgene) as described by Dull, J Virol 72:8463-8471 (1998). Control lentivirus was also made using an empty vector pLVX-IRES-puro. Ad-MEK1 CA was from Cell Biolabs, Inc. Ad-Mek1 DN was purchased from Vector Biolabs. All adenoviruses were produced, amplified and purified as described by Dull 1998; Luo, et al. Nat Protoc 2:1236-1247 (2007). For lentiviral infection, cells were incubated with lentivirus in growth medium in the presence of 8 μg/ml polybrene (Sigma) for 8 hours. For adenoviral infection, cells were incubated with adenovirus (MOI=100) in growth medium for overnight.

Quantitative RT-PCR (qPCR) and Immunoblots.

Total RNA was purified using a QiagenRNeasyPlus Mini kit. cDNA was synthesized using a Bio-Rad iScript cDNA Synthesisis kit. qPCR was performed using a Bio-Rad iQ SYBR Green Supermix on a Bio-Rad CFX96 Real Time System. Primers used for qPCR were listed in Table 1. For immunoblot, cells were lysed in RIPA buffer supplemented with protease and phosphatase inhibitors. Cell lysates were then separated with 4-20% gradient gel (Bio-Rad) and subjected to immunoblot.

Primary Mouse Endothelial Cell Isolation from Embryos.

Primary mouse endothelial cells were isolated from E12.5 embryos using a protocol similar to that described by Ren, et al. J Clin Invest 120:1217-1228 (2010). Briefly, E12.5 embryos were harvested, minced finely with scissors, and then digested in 25 ml collagenase 0.2% (w/v) at 37° C. for 20 minutes. The crude cell preparation was pelleted and resuspended in DPBS. The cell suspension was incubated with CD31-coated beads (Invitrogen) at room temperature for 10 minutes with rotation. Using a magnetic separator, the bead-bound cells were recovered, washed with DMEM-20% FBS. The cells were then used for gene expression analysis by qPCR.

### Administration of U0126.

Time-mating was set up as described above. At 13.5 days post coitum, plugged mice were treated with MEK inhibitor U0126 (5 mg/kg body weight) or same volume of DMSO dissolved in PBS by intraperitoneal injection. Twenty four hours later, E14.5 embryos were harvested, and the lymphatic vasculature in the skins were visualized by VEGFR3 whole mount staining.

### Data Analysis.

All data are presented as the Mean±SEM of at least three separate experiments. Differences between groups were tested for statistical significance using Student's t-test.

Results

### Generation of a Noonan Syndrome Mouse Model.

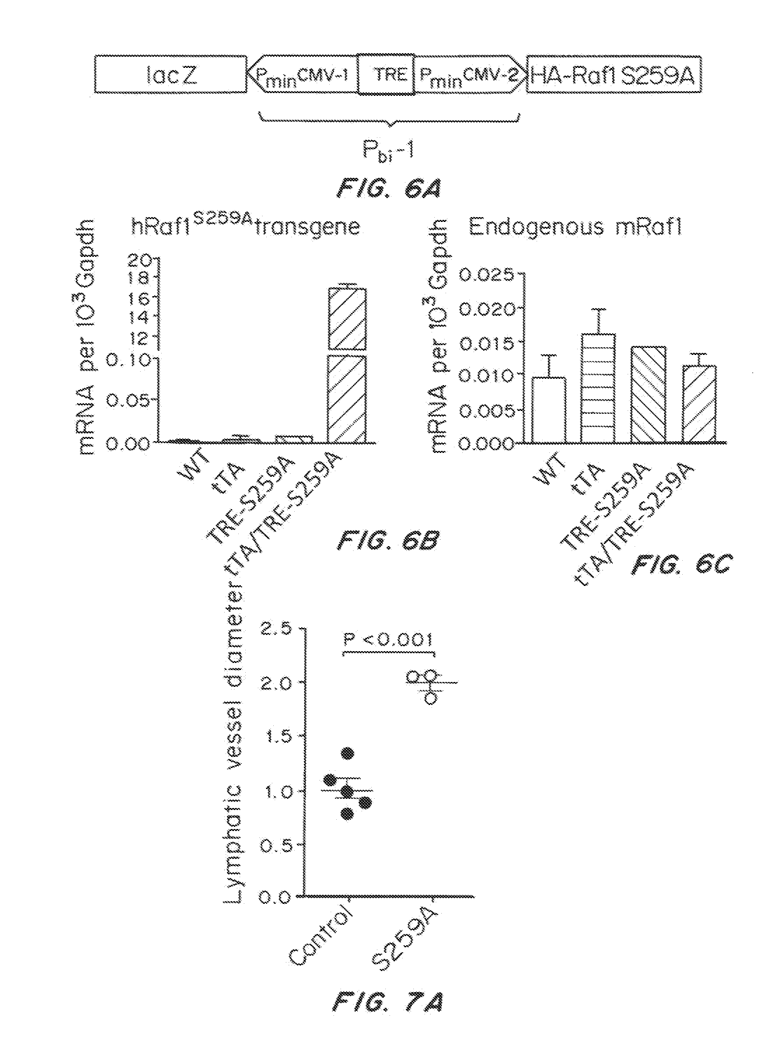

Gain-of-function RAF1 mutations such as RAF1S259A have been associated with Noonan syndrome. To explore the molecular basis of the vascular defects in Noonan syndrome, endothelial-specific RAF1S259A transgenic mice were generated by crossing a line with a bidirectional CMV promoter under control of a tetracycline-responsive promoter element driving human RAF1S259A and LacZ (TRE-RAF1S259A) (FIG. 6A) with VE-cadherin-tT A mice Sun, et al. Proc Natl Acad Sci USA 102:128-133 (2005). To confirm expression of the transgene, ECs were isolated from E12.5 embryos and analyzed for RAF1S259A expression by quantitative RT-PCR using human RAF1 specific primers (FIG. 6B). The endothelial-specific expression of the transgene was further confirmed by whole mount X-gal staining of E12.5 embryos. Of 58 pups from the TRE-RAF1S259A and VE-cadherin-tTA cross, only two VE-cadherin-tTA/RAF1S259A double transgenic (S259A) mice were born alive. X-gal staining showed that these two mice were barely stained suggesting that endothelial-expression of RAF1S259A causes embryonic lethality. At E9.5 and E10.5, only a small portion of the ECs showed positive x-gal staining By E12.5, a majority of the ECs are x-gal positive. This suggests that the VE-cadherin promoter is not fully turned on until E12.5 or later stage, which is consistent with previous observation. Prior to E12.5, no significant defects were observed in the cardiovascular system of S259A embryos. S259A embryos at E14.5 showed a gross subcutaneous edema with nearly 100% (53 of 55 embryos) lethality by E15.5. No hemorrhage was observed except for subcutaneous bleeding in the neck dorsally to the right ear in half of the embryos.

### S259A Mouse Develops Lymphangiectasia.

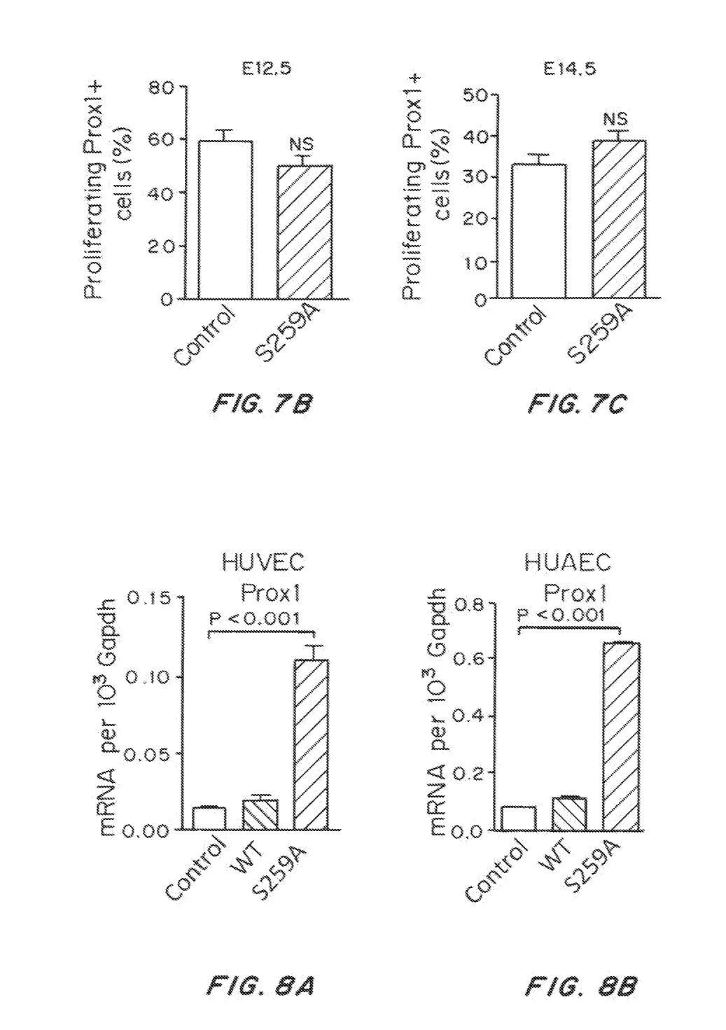

The extensive edema in S259A embryos indicates that they developed lymphatic defects. H&E and X-Gal staining of sections of E14.5 embryos revealed massive enlarged and malformed subcutaneous vessels in S259A embryos compared to that in the control littermates. VEGFR3 and PROX1 immunofluorescent staining showed that these vessels are lymphatics. A common feature was the presence of connexin 40 (CX40)- and neuropilin 1 (NRP1)-positive, VEGFR3- and PROX1-negative arteries surrounded by dilated lymphatics, a pathognomic finding in lymphangiectasia syndromes (Faul, et al. Am J Respir Crit Care Med 161:1037-1046 (2000))). The diameter of skin lymphatic vessels in E14.5 S259A embryos was on the average twice as large as that of control littermates (FIG. 7A) and the jugular lymphatic sacs were also grossly dilated. No significant difference in subcutaneous blood vessels was observed at this stage. These results suggest that endothelial expression of RAF1S259A had a more profound impact on lymphatic than on blood vessels. Together, S259A mouse develops lymphangiectasia which mimics the lymphatic phenotypes in Noonan syndrome, indicating a similar effect of RAF1S259A on the vasculature in mouse as in human patients.

### RAF1S259A Induces Lymphatic Endothelial Fate Specification.

To investigate if the lymphatics enlargement in S259A embryos was due to increased LEC proliferation, the number of Ki67⁺/PROX1⁺ cells was determined in both E12.5 or in E14.5 embryos. No significant differences from littermate controls were seen (FIGS. 7B, 7C). Thus this could be due to induced lymphatic fate specification.

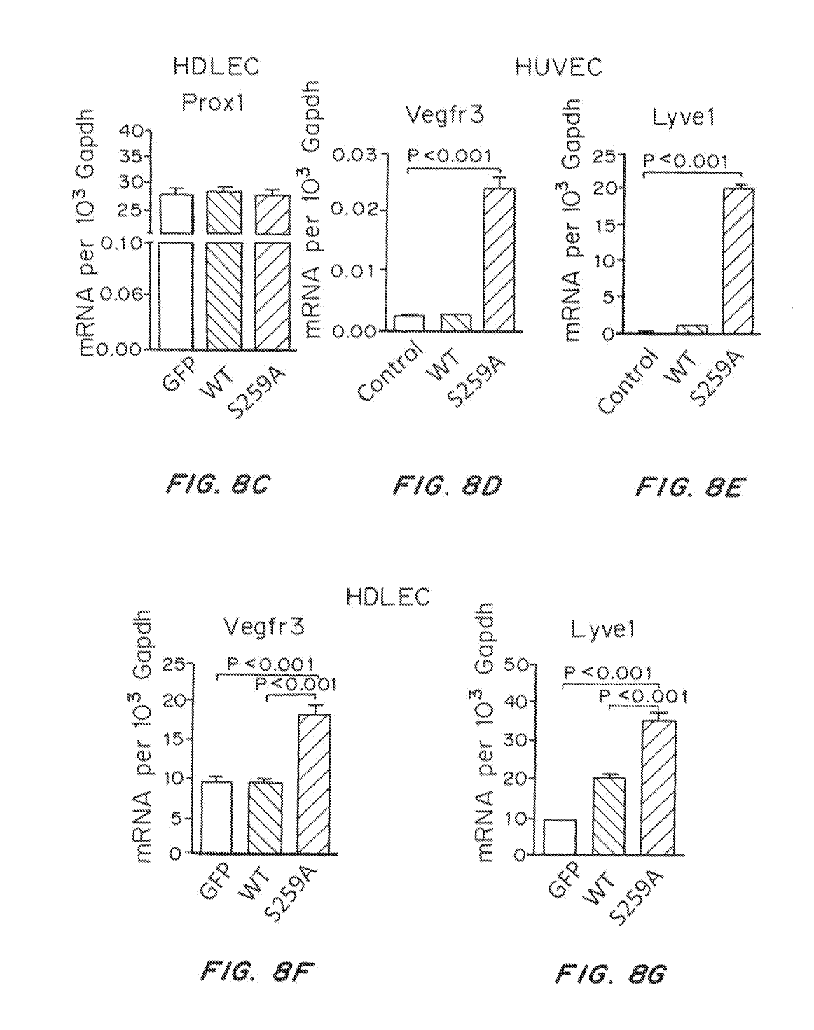

The effect of RAF1S259A on lymphatic marker expression was then examined. Expression of PROX1, a master control of lymphatic fate, was examined. At E12.5, in wild type embryos PROX1 was evident only in lymph sacs with no expression seen in the cardinal vein. In contrast, in S259A embryos PROX1 was detected in both cardinal veins and lymph sacs at a comparable expression level. By E14.5, PROX1 was still detectable in the jugular veins of S259A embryos though at a lower level compared to jugular lymph sacs. No PROX1 expression was observed in the arteries of either S259A embryos or the control littermates. No difference in PROX1 levels between β-GAL positive and negative cells was observed in lymph sacs, suggesting that RAF1S259A cannot induce PROX1 in LEC. In agreement with this data, RAF1S259A upregulated Prox1 expression both in HUVEC (FIG. 8A) and HUAEC (FIG. 8B), but not in HDLEC (FIG. 8C). Since expression of PROX1 in human blood vascular EC induces LEC differentiation (Hong, et al. Dev Dyn 225:351-357 (2002)), expression of lymphatic markers was investigated in RAF1S259A EC. Increased expression of Vegfr3 and Lyve-1 was observed in vitro and in vivo in HUVEC (FIGS. 8D, 8E), and HDLEC (FIGS. 8F, 8G).

### RAF1S259A Induces Sox18-Initiated Lymphatic Endothelial Fate Specification.

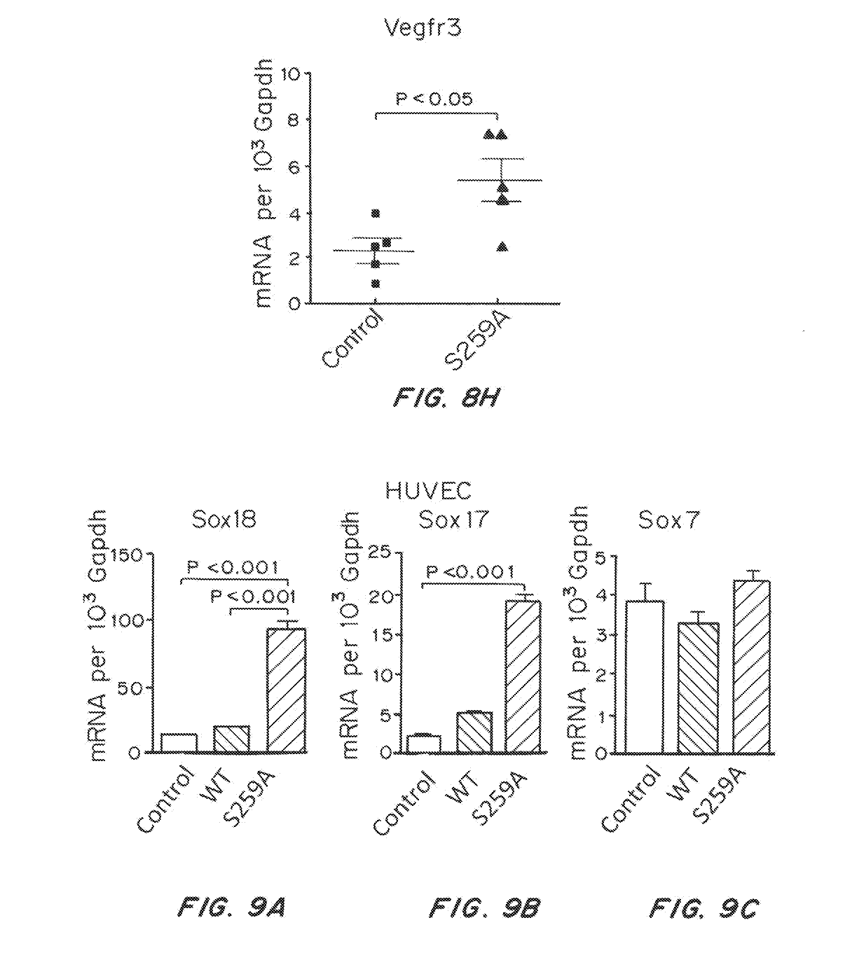

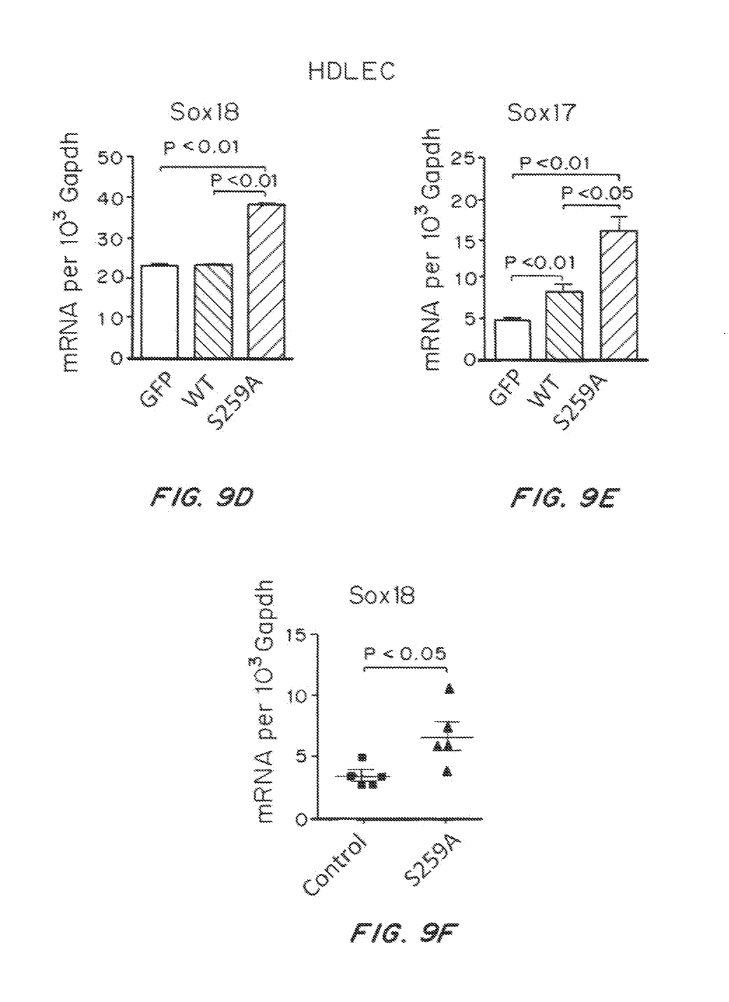

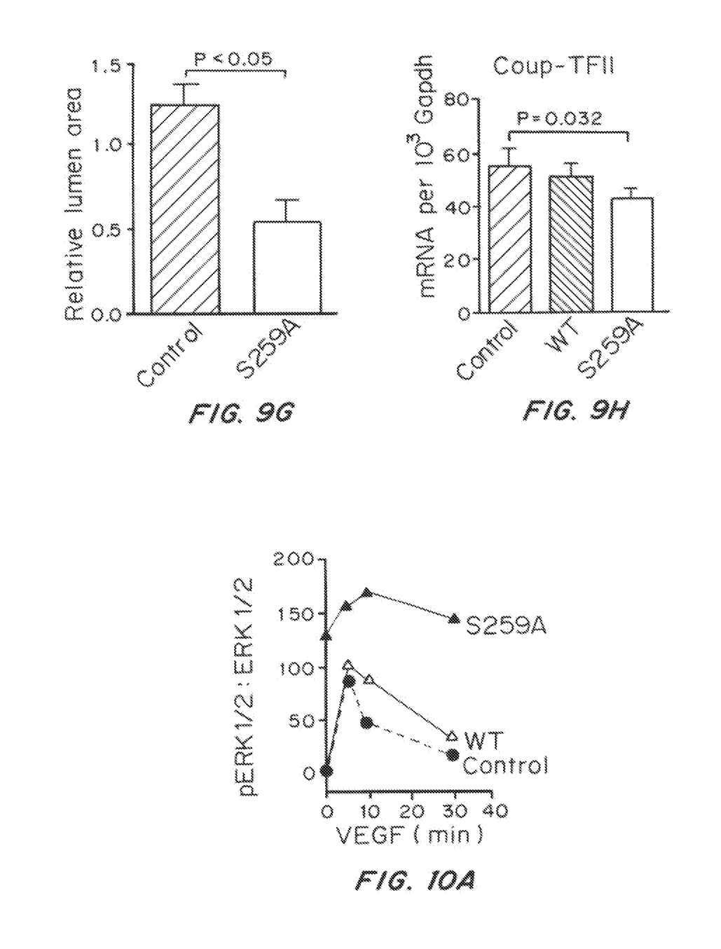

During early embryonic development, lymphatic fate specification is initiated by Sox18 which directly controls Prox1 gene expression. To study the mechanism of RAF1S259A-dependent induction of lymphatic fate, adenoviral vectors were used to study its effects in cultured EC. In HUVEC and HDLEC, expression of RAF1S259A, but not a wild type Raf1, induced a significant increase in Sox18 (FIGS. 9A, 9D) and Sox17 ((FIGS. 9B, 9E) levels while Sox7 (FIG. 9C) remained unchanged. To confirm Sox18 induction by RAF1S259A in vivo, its expression in primary EC purified from E12.5 S259A embryos was examined. In agreement with in vitro results, Sox18 levels were significantly higher compared to ECs isolated from control littermates (FIG. 8C) while immunofluorescence staining demonstrated strong SOX18 expression in β-GAL-positive cells in S259A embryos at E11.5. At E11.5, SOX18 expression was detected in the blood vasculature at E11.5 but, as expected (Francois et al. Nature 456:643-647 (2008)), it was lost by E14.5. In contrast to wild type, S259A embryos demonstrated stronger SOX18 expression in the blood vasculature at E11.5, that persisted at E14.5 both in blood and lymphatic vessels, suggesting that RAF1S259A can overcome normal downregulation of SOX18 expression.

While COUP-TFII has also been shown to be required for the initiation and early maintenance of PROX1 expression in LEC (Srinivasan, et al. Genes Dev 24:696-707 (2010), endothelial RAF 1S259A expression had no significant effect on Coup-TFII levels in vitro or in vivo (FIG. 9E). Persistent increase in SOX18 and PROX1 expression in cardinal veins of S259A embryos can be expected to result in prolonged venous EC to LEC differentiation and a continuous migration of PROX1-positive LEC to lymph sacs. Indeed, there was a larger number of migrating PROX1/SOX18 double-positive cells from cardinal veins in S259A embryos at E11.5. As a result, cardinal veins in S259A embryos by E14.5 were significantly smaller than in littermate controls (FIG. 9G).

### RAF1-AKT Crosstalk Regulates Lymphatic Endothelial Fate Specification.

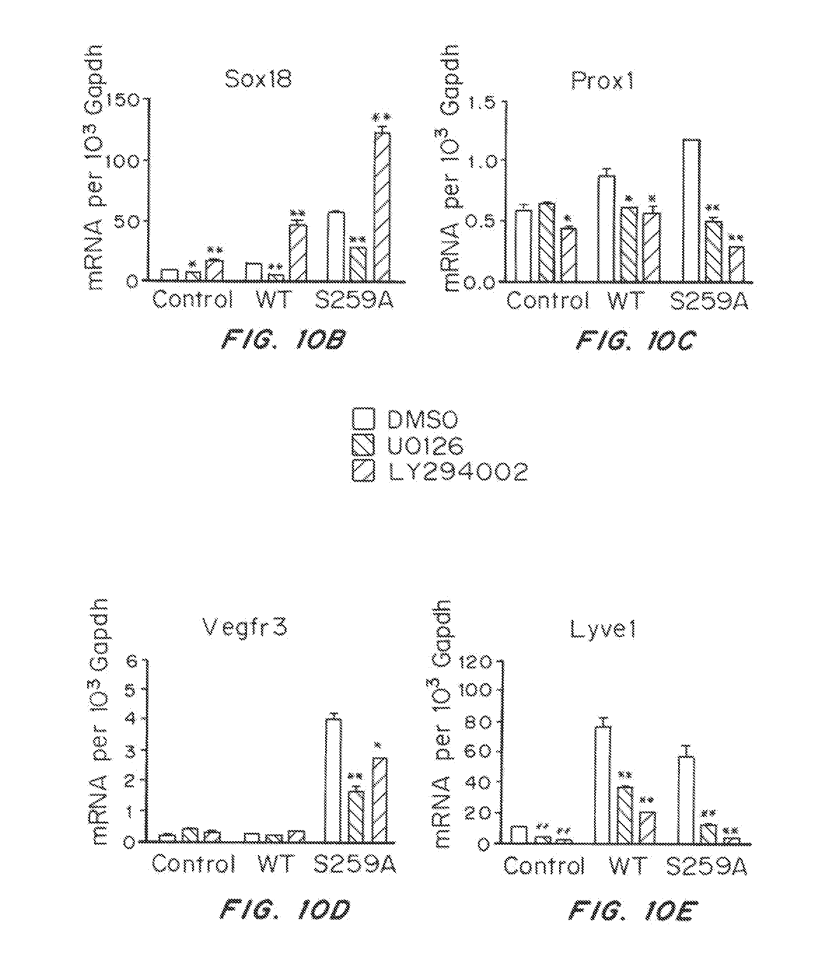

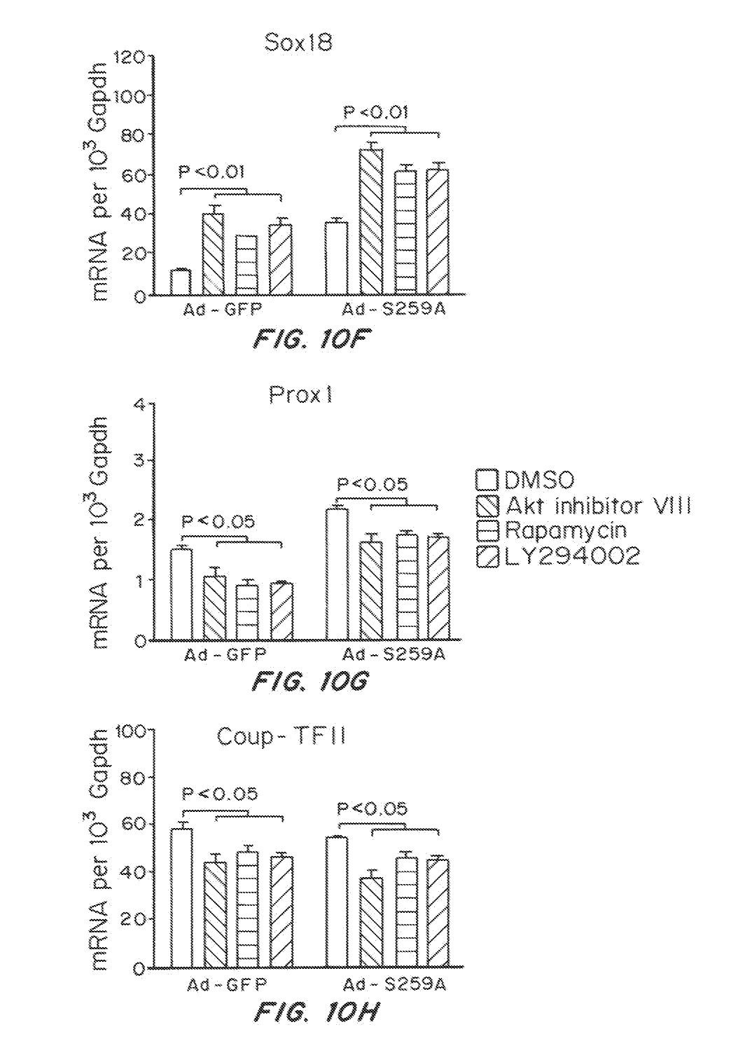

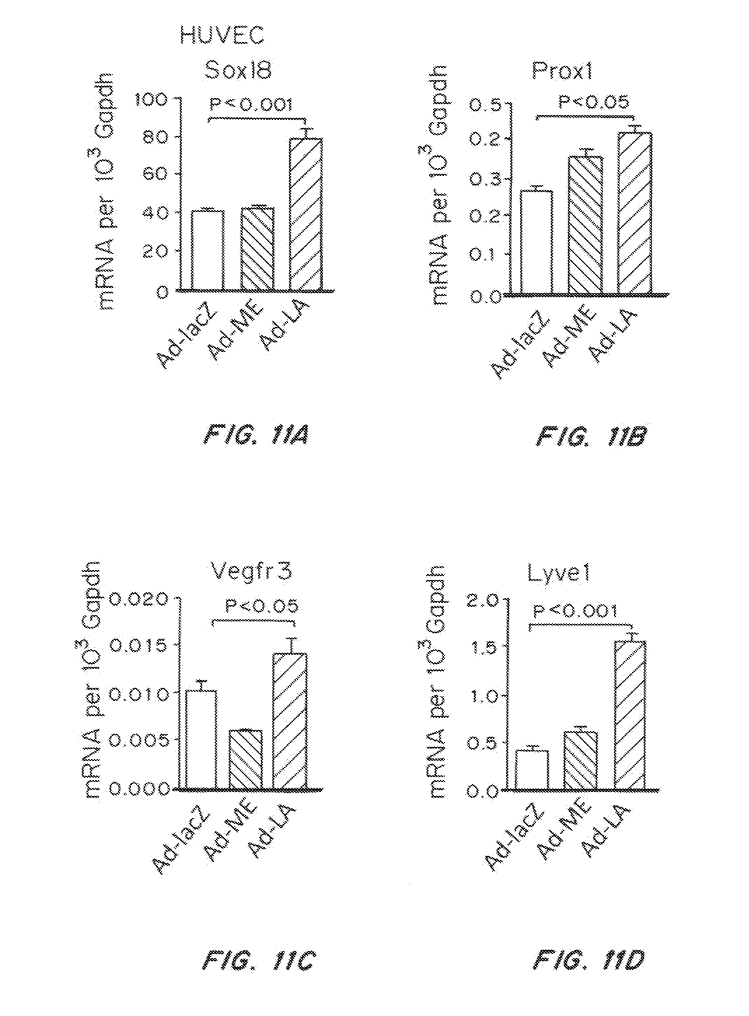

The Ser259 to Ala259 mutation of RAFT decouples the RAF1-AKT crosstalk and thus renders RAF1 resistant to inhibition by PI3K/Akt signaling. This results in simultaneous activation of both RAF1/ERK and PI3K/AKT signaling pathway, enabling further evaluation of the role of RAF1-AKT crosstalk in lymphatic fate specification. In HUVEC, RAF1S259A effectively decouples these two signaling pathways, resulting in constitutive activation of Erk while Akt signaling remains normal (FIG. 10A). ERK activation in S259A embryos was demonstrated by anti-pERK1/2 staining of β-GAL positive ECs. To determine the specific role of these two pathways in lymphatic fate specification, the effect of its inhibition on Sox18 (FIG. 10B), Prox1 (10C), Vegfr3 (FIG. 10D) and Lyve1 (FIG. 10E) expression was determined. Shutdown of RAF1/MEK/ERK pathway by MEK inhibitor U0126 blocked induction of all four genes by RAF1S259A in vitro (FIGS. 10B-10E). In contrast, inhibition of PI3K/AKT signaling led to an increase in Sox18 expression, likely due to baseline AKT suppression of RAF1 activation. At the same time, expression of Prox1, Vegfr3 and Lyve1 was significantly reduced by PI3K inhibition. Consistently, inhibition of mTOR by rapamycin or direct inhibition of AKT activation by AKT inhibitor VIII also significantly inhibited Prox1 (FIG. 10G) and Coup-TFII (FIG. 10H) expression while slightly inducing Sox18 expression (FIG. 10F). These results suggest Sox18 expression is exclusively dependent on RAF1/MEK/ERK signaling while Prox1, Vegfr3 and Lyve1 expression requires both MEK/ERK and PI3K/AKT signaling inputs.

### Excessive ERK Activation is the Basis of Lymphatic Abnormalities in Noonan Syndrome.

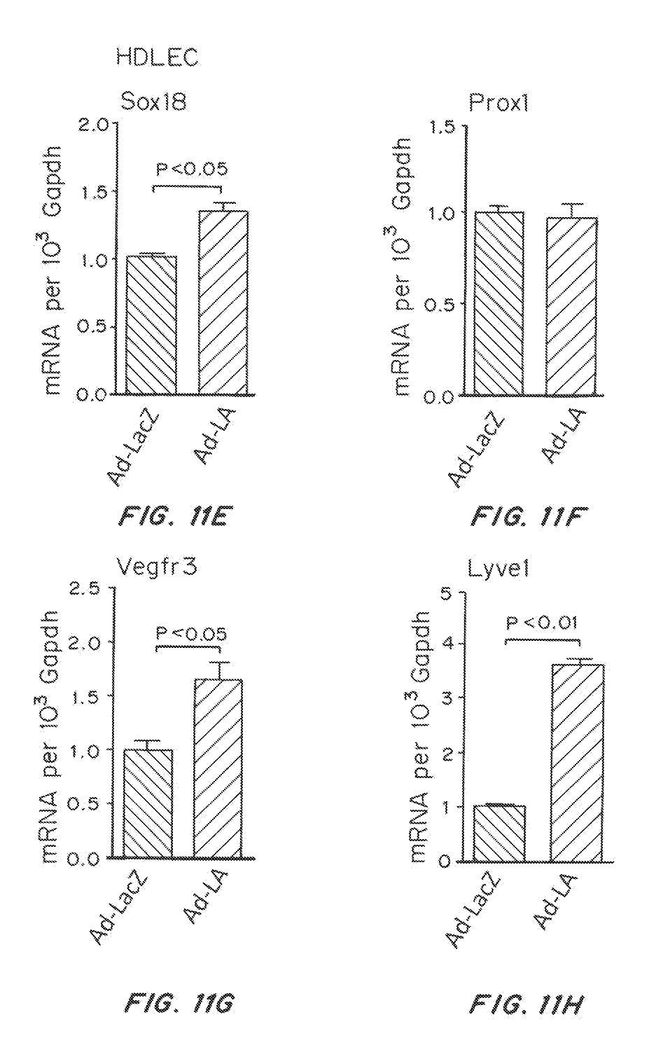

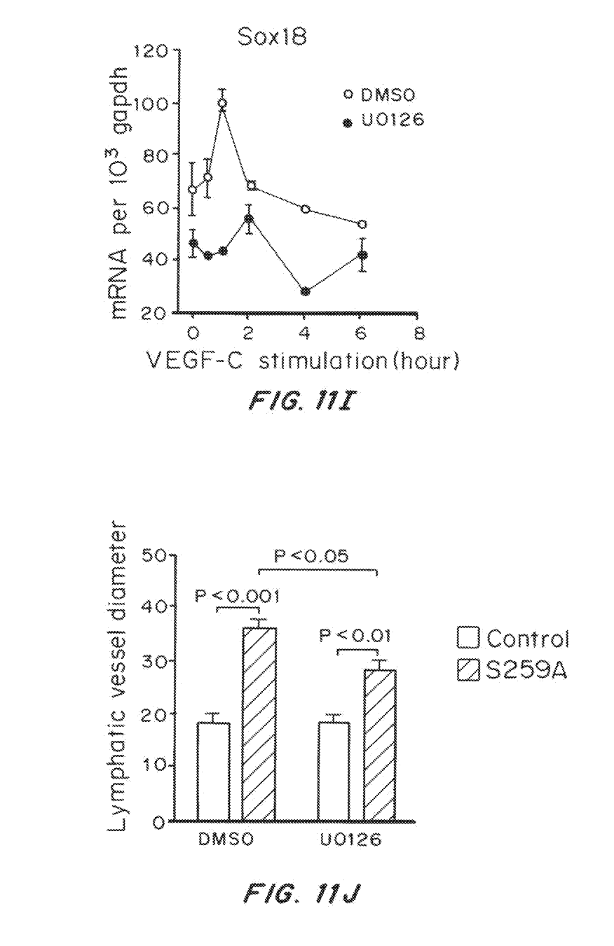

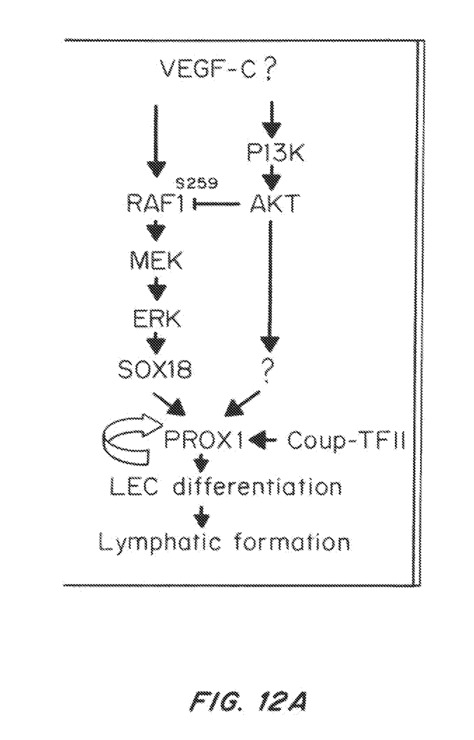

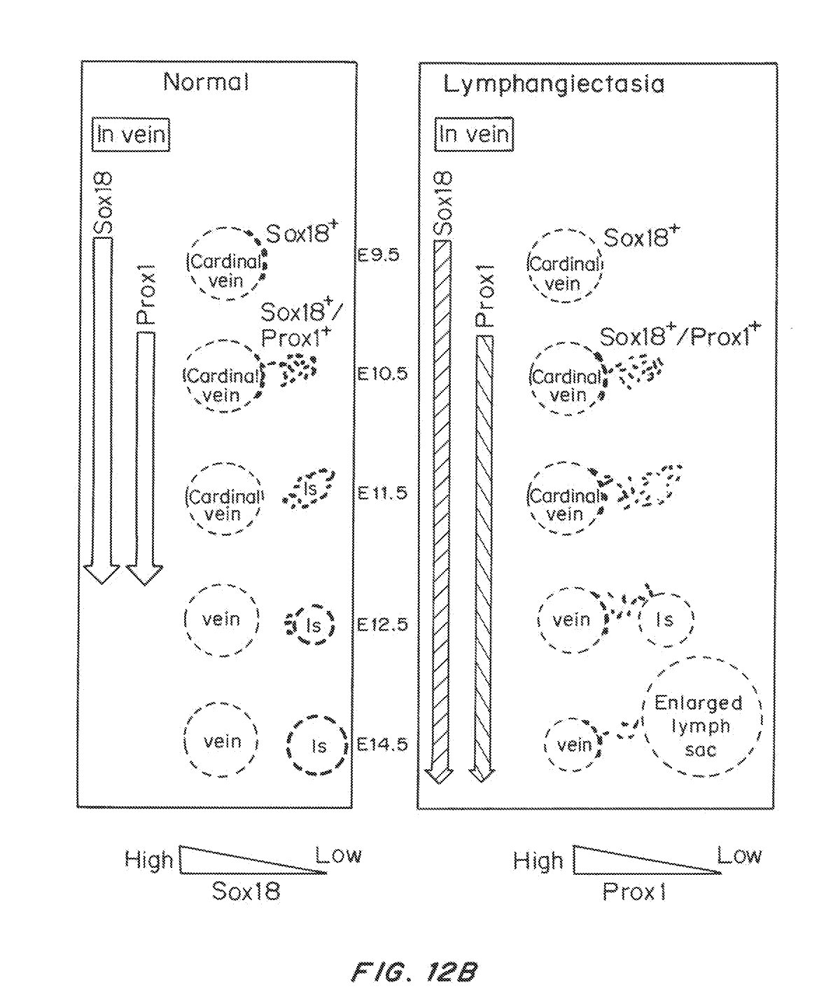

ERK signaling is constitutively activated by RAF in ECs, and is essential for lymphatic fate induction. It was then determined whether ERK activation is sufficient to cause the lymphatic phenotypes in S259A embryos by expression of constitutively active ERK constructs. In agreement with the above observations, expression of a nuclear (LA) but not a cytoplasm-localized (ME) constitutively active ERK construct (Ren, et al. J Clin Invest 120:1217-1228 (2010) induced expression of Sox18, Vegfr3 and Lyve1 in both HUVEC (FIGS. 11A-11D) and HDLEC (FIGS. 11E-11H). Prox1 was induced only in HUVEC but not in HDLEC (FIGS. 11C, 11G), in agreement with blood EC (BEC)-specific induction of Prox1 by RAF1S259A. These results suggest that ERK activation is sufficient for induction of Sox18, Vegfr3 and Lyve1 in both BECs and LEC, while Prox1 induction is specific to BEC.

Given the essential role of VEGF-C signaling in lymphatic development, ERK-dependence of its effect on Sox18 in HDLEC was analyzed. VEGF-C induced Sox18 expression was completely abolished by inhibition of MEK/ERK signaling (FIG. 11I). To rule out the paracrine effect of ERK induced Sox18 expression through VEGFs, Vegf-a, c and d expression was examined after Sox18 knocking down. No differences were found in HUVEC and primary mouse venous ECs in Sox18 knocked down cells compared to control cells. Whether inhibition of MEK/ERK signaling can rescue the lymphatic phenotype in S259A embryos was determined. Intraperitoneal injection of U0126 into pregnant mice at E13.5, the time when lymphatic network is forming in the skin of mouse embryos, led to a significant decrease in lymphangiectasia in S259A embryos 24 hrs later (FIG. 11J). Together, these results suggest that constitutive ERK activation induced excessive lymphatic fate specification is the basis of lymphatic abnormalities in Noonan syndrome.

In summary, the data supports a model in which RAF1-MEK-ERK signaling induces LEC fate specification and lymphatic vessel development by controlling SOX18 expression (FIG. 12A). Persistent ERK activation leads to a prolonged expression of Sox18 and Prox1 in venous EC, resulting in excessive transition of these venous EC to the lymphatic fate that, in turn, leads to increased outmigration of these newly specified LEC to the forming lymphatic sacs. The sacs are greatly increased in size and give rise to disproportionally large lymphatics thus leading to lymphangiectasia (FIG. 12B).

The morphologic features observed in RAF1S259A transgenics include large, irregularly shaped lymphatic vessels wrapping around arterioles and small arteries, a finding pathognomonic to patients with Noonan's syndrome, and extensive edema that is the likely cause of the death in these embryos. At the same time, with the exception of the right jugular sac in about 50% of the embryos where some hemorrhage was observed, no blood was observed anywhere else in any of the lymphatics suggesting complete separation of venous and lymphatic circulations.

VEGF-A (a principal VEGF in arterial and venous endothelial cells (EC)) and VEGF-C (the key VEGF in lymphatic EC) via their receptor VEGFR2 and VEGFR3 (FLT4) activate several signaling cascades including PI3K/AKT and RAS/RAF/ERK. Until recently, no interactions between these two signaling cascades have been appreciated. Under normal conditions in adult vasculature, AKT inhibits ERK signaling via phosphorylation of RAF1, and that inhibition of PI3K leads to activation of ERK (Ren et al. 2010). The importance of PI3K/ERK interaction is suggested by studies demonstrating that mice lacking the pik3r1 regulatory subunit display defects in lymphatic remodeling and maturation while lymphatic vessels display upregulation of BEC markers such as endoglin (Mouta-Bellum, et al. Dev Dyn 238:2670-2679 (2009)).

The role of PI3K/AKT-RAF1/MEK/ERK cross-talk in the developing vasculature using a RAF1 mutant insensitive to AKT phosphorylation was determined. Since the promoter construct used in these studies results in activation of the mutant construct expression at approximately E9.5, the time of lymphatic fate specification, the effect of this mutation on lymphangiogenesis was studies. Decoupling of PI3K and ERK pathways in vitro resulted in activation of ERK even in the absence of VEGF (or other growth factor) stimulation, demonstrating that the former tonically suppresses the latter. In vivo this resulted in activation of Sox18 expression in cardinal veins and led to induction of PROX1 expression that, in turn, initiated lymphangiogenesis. These results strongly argue that ERK controls SOX18 expression and that transient activation of ERK signaling, perhaps due to inhibition of AKT signaling input, is responsible for lymphatic fate specification.

While ERK can induce SOX18 expression in venous, arterial and lymphatic EC, PROX1 expression increased only in venous EC. This is consistent with the known requirement of COUP-IIF for PROX1 expression. During normal development in vivo, PROX1 induction is limited to the basolateral aspect of cardinal veins, demonstrating that it is not only venous EC-specific, but also a spatial context-dependent process. Introduction of the mutant RAF 1S259A construct induced Sox18 expression throughout the vein but the spatial regulation of PROX1 induction was, nevertheless, maintained.

The event responsible for the induction of ERK signaling in the cardinal vein is not certain. Both VEGF-A and VEGF-C have the ability to activate PLCγ/ERK pathway acting, respectively, via VEGFR2 and VEGFR3. Furthermore, both receptors are expressed in the cardinal vein at that moment in time and the data show that VEGF-C can induce Sox18 expression in an ERK-dependent manner in vitro. At the same time, previous studies have shown that while VEGF-C is essential for the formation of lymphatic sprouts from embryonic veins, it is dispensable for cell commitment to the lymphatic endothelial lineage. Furthermore, mice with VEGFR3 loss-of-ligand-binding mutation show defective lymphatic vessel growth, while jugular lymph sacs develop normally. Taken together, this suggests that VEGF-A, and not VEGF-C, may be responsible for the lymphatic fate commitment. It should also be noted that ERK activation in RAF1S259A mutant embryos is due to removal of baseline AKT inhibition of RAF1 and not a direct activating effect of the transgene itself. Thus, induction of Sox18 may as much depend on the withdrawal of AKT activation as on direct activation of RAF/ERK pathway.

Recent studies have shown that RAF1 functions in both kinase dependent and independent manner, with the latter not requiring ERK activation (Mielgo, et al. Nat Med 17:1641-1645 (2011). The lymphatic phenotype in S259A embryos is due to RAF1-dependent activation of ERK since expression of wild type RAF1 in EC has no significant effects on Sox18, Prox1 and other LEC markers were observed. At the same time, introduction of a constitutively active ERK construct (LA) had a similar effect on LEC specification to RAF1S259A. In addition, inhibition of ERK activation by a MEK inhibitor blocked RAF1S259A induction of SOX18 expression. Overall, these results indicate that RAF1S259A induces lymphatic specification in an ERK-dependent manner.

Gain-of-function mutations in the RAS signaling cascade, including those of RAF1, have been shown to cause Noonan syndrome and related disorders. Pandit, et al. Nat Genet 39:1007-1012 (2007); Razzaque, et al. Nat Genet 39:1013-1017 (2007)). Common features of these “RASopathies” include increased ERK activation and lymphangiectasia (Ozturk, et al. Int J Clin Pract 54:274-276 (2000); Herzog, J Pediatr 88:270-272 (1976)). The data showed that endothelial-specific expression of RAF1S259A causes a lymphatic phenotype similar to that observed in Noonan's and LEOPARD patients. Furthermore, inhibition of ERK activation in vivo reversed lymphangiectasia formation in S259A mice. Thus, endothelial ERK activation is responsible for the lymphatic phenotype observed in these patients and suppression of ERK activation should be of therapeutic benefit.

In summary, the essential role of endothelial ERK signaling in lymphatic fate determination via induction of Sox18 expression in cardinal veins. Excessive ERK activation, induced by Raf1 activating mutations induce excessive venous to lymphatic fate shift and are the molecular basis of lymphangiectasia and other lymphatic abnormalities seen in various “RASopathy” syndromes.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treating defects, disorders or diseases of insufficient blood or lymphatic vasculature comprising administering to a site in a patient in need thereof, a pharmaceutical composition comprising a molecule specifically blocking RAF1-AKT crosstalk in pharmaceutically acceptable carrier or excipient in an amount effective to enhance the growth of blood or lymphatic vasculature at the site.

2. The method of claim 1 comprising reducing the bioavailability of RAF 1.

3. The method of claim 1 comprising blocking the phosphorylation of RAF1 by AKT1.

4. The method of claim 1 comprising administering small molecules, nucleic acids or antibodies specifically binding to AKT1 or RAF1, or blocking expression or translation thereof, to block RAF1-AKT crosstalk.

5. The method of claim 1, wherein the molecule is a nucleic acid encoding RAF1 S259A, RAF1S259A, or a molecule which increases RAF1 S259A bioavailability.

6. The method of claim 1, wherein the molecule is released from a device, nano or microparticles or controlled or sustained release formulation.

7. The method of claim 1, wherein the pharmaceutical composition is administered by an intracoronary, intramuscular, intraarterial, intravenous, intraperitoneal or subcutaneous route.

8. The method of claim 1 wherein the patient is in need of lymphatic vasculature.

9. The method of claim 1, wherein the patient is in need of blood vasculature.

10. The method of claim 1 wherein the patient suffers from a condition associated with defective arterial development or arterial insufficiency.

11. The method of claim 1 comprising administering the formulation to a patient at the time of, or immediately before or after, implantation of a device, graft or transplant.

12. A formulation for use in the method of claim 1 comprising a molecule specifically blocking RAF1-AKT crosstalk in a pharmaceutically acceptable carrier or excipient in an amount effective to enhance the growth of blood or lymphatic vasculature at the site.

13. A device, nano or microparticles or controlled or sustained release formulation comprising RAF1-AKT blocking molecules.

14. The device, nano or microparticles or controlled or sustained release formulation of claim 13 comprising small molecules, nucleic acids or antibodies specifically binding to AKT1 or RAF1, or blocking expression or translation thereof, to block RAF1-AKT crosstalk.

15. The device, nano or microparticles or controlled or sustained release formulation of claim 13 comprising a nucleic acid encoding RAF1 S259A, RAF1S259A, or a molecule which increases RAF1 S259A bioavailability.

16. The device, nano or microparticles or controlled or sustained release formulation of claim 13, comprising an AKT1 inhibitory molecule blocking phosphorylation of RAF1.

17. The device, nano or microparticles or controlled or sustained release formulation of claim 16, wherein the AKT1 inhibitory molecule is selected from the group consisting of an siRNA, an antibody specific for AKT1, an antisense oligonucleotide, a ribozyme or a small molecule.

18. A method of treating defects, disorders or diseases of insufficient blood or lymphatic vasculature comprising administering to a site in a patient in need thereof, a pharmaceutical composition increasing expression of ERK at the site in need thereof for period of time effective to enhance the growth of blood or lymphatic vasculature at the site in the patient in need thereof.

19. The method of claim 18 wherein the pharmaceutical composition comprises a nucleic acid molecule expressing ERK incorporated into or onto a device, nano or microparticles or controlled or sustained release formulation for administration at the site in need thereof.

20. A nucleic acid molecule expressing ERK incorporated into or onto a device, nano or microparticles or controlled or sustained release formulation for administration at a site in need thereof.


SEQUENCE LISTING (26 sequences)

SEQ ID NO: 1 - DNA, 19 bp, Artificial Sequence
  synthetic construct; hSox18 primer
cttcatggtgtgggcaaag

SEQ ID NO: 2 - DNA, 18 bp, Artificial Sequence
  synthetic construct; hSox18 primer
gcgttcagctccttccac

SEQ ID NO: 3 - DNA, 20 bp, Artificial Sequence
  synthetic construct; hSox7 primer
cagcaagatgctgggaaagt

SEQ ID NO: 4 - DNA, 20 bp, Artificial Sequence
  synthetic construct; hSox 7 primer
gttggggtagtcctgcatgt

SEQ ID NO: 5 - DNA, 20 bp, Artificial Sequence
  synthetic construct; hGAPDH primer
tgcaccaccaactgcttagc

SEQ ID NO: 6 - DNA, 21 bp, Artificial Sequence
  synthetic construct; hGAPDH primer
ggcatggactgtggtcatgag

SEQ ID NO: 7 - DNA, 20 bp, Artificial Sequence
  synthetic construct; hCoup-TFII primer
gcaagtggagaagctcaagg

SEQ ID NO: 8 - DNA, 20 bp, Artificial Sequence
  synthetic construct; hCoup-TF-II primer
gctttccacatgggctacat

SEQ ID NO: 9 - DNA, 20 bp, Artificial Sequence
  synthetic construct; hLyvel primer
acttccatctggaccacgag

SEQ ID NO: 10 - DNA, 20 bp, Artificial Sequence
  synthetic construct; hLyvel primer
agcctacaggcctccttagc

SEQ ID NO: 11 - DNA, 20 bp, Artificial Sequence
  synthetic construct; hSox17 primer
cagaatccagacctgcacaa

SEQ ID NO: 12 - DNA, 19 bp, Artificial Sequence
  synthetic construct; hSox17 primer
gcggccggtacttgtagtt

SEQ ID NO: 13 - DNA, 20 bp, Artificial Sequence
  synthetic construct; hProx1 primer
ggcattgaaaaactcccgta

SEQ ID NO: 14 - DNA, 20 bp, Artificial Sequence
  synthetic construct; hProx1 primer
acagggctctgaacatgcac

SEQ ID NO: 15 - DNA, 20 bp, Artificial Sequence
  synthetic construct; hVEGFR3 primer
ggtgtcgatgacgtgtgact

SEQ ID NO: 16 - DNA, 18 bp, Artificial Sequence
  synthetic construct; hVEGFR3 primer
ctctgcctgggactcctg

SEQ ID NO: 17 - DNA, 20 bp, Artificial Sequence
  synthetic construct; hRaf1 primer
acccattcagtttccagtcg

SEQ ID NO: 18 - DNA, 20 bp, Artificial Sequence
  synthetic construct; hRaf1 primer
gctaccagcctcttcattgc

SEQ ID NO: 19 - DNA, 20 bp, Artificial Sequence
  synthetic construct; mRaf1 primer
caagtggcatggagatgttg

SEQ ID NO: 20 - DNA, 20 bp, Artificial Sequence
  synthetic construct; mRaf1 primer
cgattgtgactcagtggtgt

SEQ ID NO: 21 - DNA, 20 bp, Artificial Sequence
  synthetic construct; mSox18 primer
aacaaaatccggatctgcac

SEQ ID NO: 22 - DNA, 20 bp, Artificial Sequence
  synthetic construct; mSox18 primer
cgaggccggtacttgtagtt

SEQ ID NO: 23 - DNA, 20 bp, Artificial Sequence
  synthetic construct; mVEGFR3 primer
gctgttggttggagagaagc

SEQ ID NO: 24 - DNA, 20 bp, Artificial Sequence
  synthetic construct; mVEGFR3 primer
tgctggagagttctgtgtgg

SEQ ID NO: 25 - DNA, 20 bp, Artificial Sequence
  synthetic construct; mGAPDH primer
aactttggcattgtggaagg

SEQ ID NO: 26 - DNA, 20 bp, Artificial Sequence
  synthetic construct; mGAPDH primer
acacattgggggtaggaaca